(19) United States
(12) Patent Application Publication
(10) Pub. No.: US 2026/0115491 A1
(43) Pub. Date: Apr. 30, 2026

(54) AURICULAR VAGAL NERVE STIMULATION TO MITIGATE INFLAMMATORY COMPLICATIONS FOR BIOACTIVE AGENTS

(71) Applicant: Aurenar, Inc., St. Louis, MO (US)

(72) Inventors: Jesse Wheeler, St. Louis, MO (US); Eric Leuthardt, St. Louis, MO (US); Jenna Gorlewicz, St. Louis, MO (US)

(73) Assignee: Aurenar, Inc., St. Louis, MO (US)

(21) Appl. No.: 19/374,198

(22) Filed: Oct. 30, 2025

Related U.S. Application Data
(60) Provisional application No. 63/713,773, filed on Oct. 30, 2024.

(51) Int. Cl. A61N 5/06; A61K 31/155; A61K 31/426; A61K 31/64; A61K 40/11; A61K 40/31; A61N 1/36

(52) U.S. Cl. CPC A61N 5/0622; A61K 31/155; A61K 31/426; A61K 31/64; A61K 40/11; A61K 40/31; A61N 1/36014

(57) ABSTRACT

Methods for treating patients receiving a medication are disclosed. A patient is administered a medication with a propensity to cause inflammation or inflammation-related side effects, or actually experiences inflammation or inflammation-related side effects in response to receiving a medication. In response, vagus nerve stimulation is provided to the patient to relieve the inflammation or inflammation-related side-effect(s). The vagus nerve stimulation may be delivered by a transcutaneous system. Any of mechanical, optical, and/or electrical energy, pulses, power, etc. may be used to target the vagus nerve. Some examples deliver the stimulation to the auricular branch of the vagus nerve.

### CROSS REFERENCE TO RELATED APPLICATIONS

The present application claims the benefit of and priority to U.S. Provisional Patent Application 63/713,773, filed Oct. 30, 2024, titled AURICULAR VAGAL NERVE STIMULATION TO MITIGATE INFLAMMATORY COMPLICATIONS FOR BIOACTIVE AGENTS, the disclosure of which is incorporated herein by reference.

### BACKGROUND

A range of therapy products, including certain drugs, gene therapies, antibodies and/or antibiotics, and cellular therapies offer promise of clinical benefits. Several such therapies have shown a propensity to inflammatory side effects. Adjunct therapies that can be offered along with the therapy products to prevent such inflammatory side effects are desired.

### SUMMARY

The present inventors have identified auricular vagal nerve stimulation, delivered non-invasively, as a potential adjunct therapy that may be beneficially provided to patients taking certain therapy products having inflammatory side effects.

A first illustrative and non-limiting example takes the form of a method of treating a patient comprising: administering to the patient a medication; observing an inflammatory response or inflammation-related side effect occurring after administering to the patient the medication; and in response to the observing, delivering a vagus nerve therapy to the patient to alleviate the observed inflammatory response or inflammation-related side effect.

A second illustrative and non-limiting example takes the form of a method of treating a patient comprising: administering to the patient a medication, the medication having potential for inflammation related side effects; and in response to the administering, initiating a regimen of vagus nerve therapy to the patient to prevent or alleviate the inflammation related side effect.

A third illustrative and non-limiting example takes the form of a method of treating a patient, comprising: administering to the patient a medication, the medication having potential for inflammation related side effects; after administering the medication at a first dosage, changing the dosage and administering the medication at a second dosage; and in response to changing the dosage and administering at the second dosage, initiating a regimen of vagus nerve therapy to the patient to prevent or alleviate the inflammation related side effect.

Additionally or alternatively to the preceding examples, the regimen may include at least one session of vagus nerve stimulation daily for a period of at least seven days. Additionally or alternatively to the preceding examples, the regimen may include two sessions of vagus nerve stimulation daily for a period of ten to fourteen days.

Additionally or alternatively to the preceding examples, the vagus nerve therapy may be delivered transcutaneously. Additionally or alternatively to the preceding examples, the vagus nerve therapy may be delivered to stimulate an auricular branch of the vagus nerve.

Additionally or alternatively to the preceding examples, the vagus nerve therapy may be mechanical. Additionally or alternatively to the preceding examples, the vagus nerve therapy may be optical. Additionally or alternatively to the preceding examples, the vagus nerve therapy may be electrical.

Additionally or alternatively to the preceding examples, the medication may be a statin. Additionally or alternatively to the preceding examples, the medication may be a chemotherapy agent. Additionally or alternatively to the preceding examples, the medication may be an immune checkpoint inhibitor. Additionally or alternatively to the preceding examples, the medication may be an antibiotic. Additionally or alternatively to the preceding examples, the medication may be a non-steriodal anti-inflammatory drug. Additionally or alternatively to the preceding examples, the medication may be allopurinol. Additionally or alternatively to the preceding examples, the medication may be an antipsychotic medication.

Additionally or alternatively to the preceding examples, the medication may be a hormonal treatment. Additionally or alternatively to the preceding examples, the medication may be a treatment for Alzheimer's disease targeting amyloid plaques. Additionally or alternatively to the preceding examples, the medication may be a Chimeric Antigen Receptor T-cell therapy. Additionally or alternatively to the preceding examples, the medication may be a diabetes drug.

Additionally or alternatively to the preceding examples, the medication may be a thiazolidinedione drug. Additionally or alternatively to the preceding examples, the medication may be an SGLT2 Inhibitor. Additionally or alternatively to the preceding examples, the medication may be a DPP-4 Inhibitor. Additionally or alternatively to the preceding examples, the medication may be sulfonylurea. Additionally or alternatively to the preceding examples, the medication may be metformin. Additionally or alternatively to the preceding examples, the medication may be a GLP-1 Receptor Agonist.

This overview is intended to introduce the subject matter of the present patent application. It is not intended to provide an exclusive or exhaustive explanation. The detailed description is included to provide further information about the present patent application.

### BRIEF DESCRIPTION OF THE DRAWINGS

The drawings illustrate, by way of example, but not by way of limitation, various embodiments discussed herein. In the drawings, which are not necessarily drawn to scale, like numerals may describe similar components in different views.

FIG. 1A is a sketch of the human ear;

FIGS. 1B-1F illustrate responses to vagus nerve stimulation in several contexts;

FIGS. 2A-2F are views of a first wearable vagus nerve modulation device;

FIG. 3 is a view of a second wearable vagus nerve modulation device;

FIGS. 4A-4B are views of a third wearable vagus nerve modulation device;

FIG. 5 is a view of a fourth wearable vagus nerve modulation device;

FIG. 6 illustrates placement of a wearable vagus nerve modulation device;

FIGS. 7-8 are side section views illustrating electrode contact to the ear;

FIG. 9 shows an illustrative example of status and warning lights;

FIGS. 10A-10G show illustrative stimulation device designs;

FIG. 11 is a block diagram for illustrative circuitry;

FIGS. 12A-12B show a block process flow diagram of an illustrative method;

FIGS. 13-14 illustrate wearable devices and charging systems;

FIG. 15 illustrates various electrode combinations that may be used;

FIGS. 16-18 illustrate further alternative designs for wearable vagus nerve modulation devices;

FIGS. 19-20 show illustrative gel pad designs; and

FIGS. 21A-21C are views of a fifth wearable vagus nerve modulation device.

### DETAILED DESCRIPTION

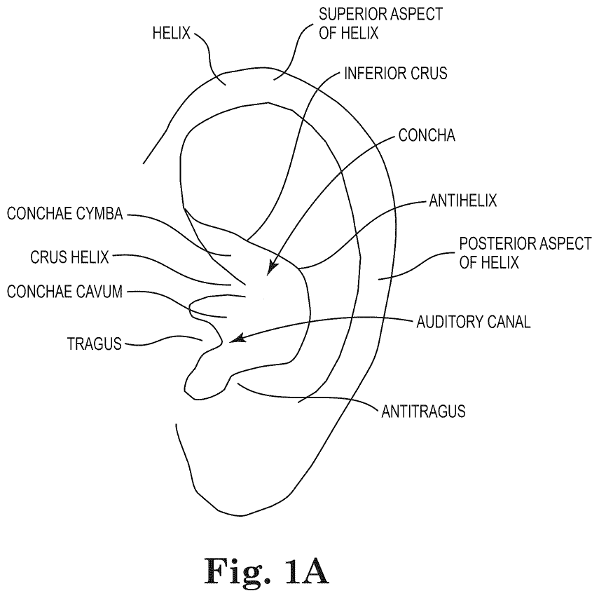

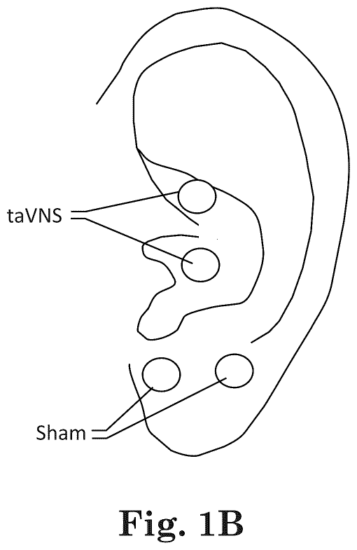

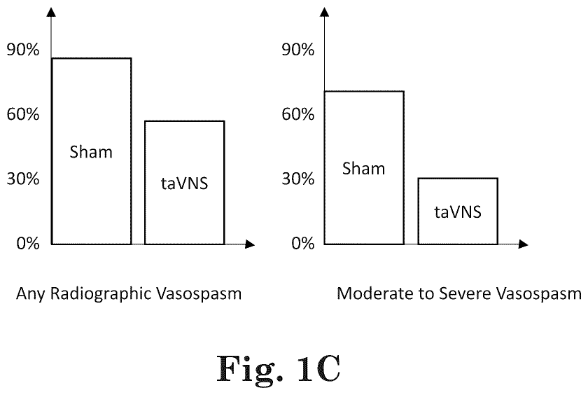

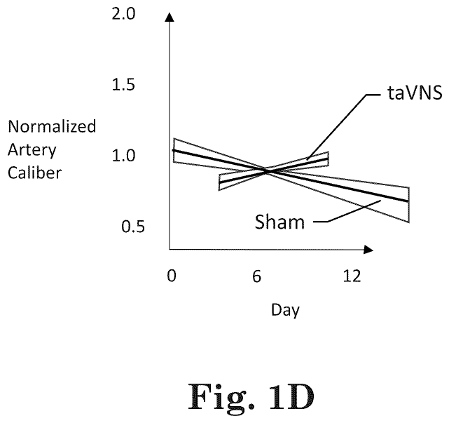

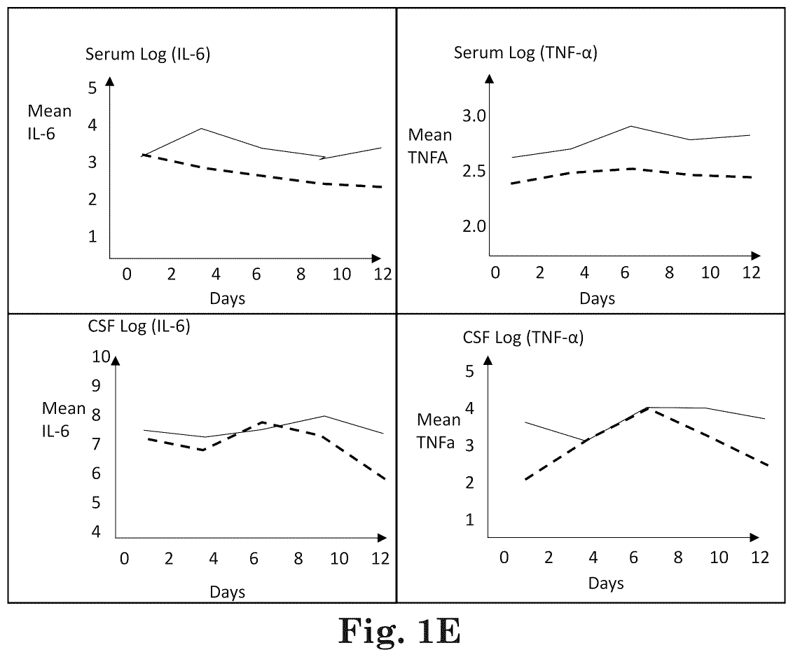

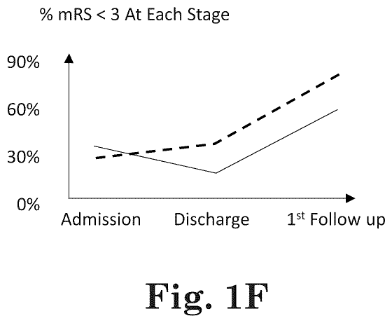

FIG. 1A is a sketch of the human ear. The auditory canal is covered at its opening by the tragus, and opens adjacent the concha. The concha is typically bisected by the crus helix into the conchae cymba superiorly and conchae cavum inferiorly. The helix is the outer rim of the ear that extends from the superior insertion of the ear on the scalp to the termination of the cartilage at the earlobe, having a superior aspect and posterior aspect, as marked in the drawing. The border of the helix usually forms a rolled rim, but the helix is highly variable in shape. The crus helix is the continuation of the anteroinferior ascending portion of the helix, and as shown in the drawing, extends in a posteroinferior direction into the cavity of the concha, typically about one half to two thirds the distance across the concha. The concha is generally bordered by the antihelix superiorly and antitragus inferiorly.

The auditory branch of the vagus nerve terminates in the crus helix. Because the vagus nerve can respond to stimuli (electrical, vibratory, acoustic, thermal, magnetic, optical, etc.) in desirable ways, any access point where such stimuli can be delivered is of great interest. Solutions for reliable, and preferably non-invasive access are in demand. Embodiments disclosed herein address shortcomings of prior approaches, as further described below.

A used herein, the phrase “nerve modulation” refers to the use of a signal, such as an electrical pulse, pulse train or other electrical signal, or any of a vibratory, acoustic, thermal, magnetic, or optical signals, to affect the nerve itself, processes the nerve controls or influences, and/or structures to which the nerve leads. More than one modality may be combined together, such as using optical and electrical stimulation together. Combinations may include alternating between two modalities, or using two modalities directed at a single volume of tissue, or directing one modality at one volume of tissue or target, while (or interleaved with) directing another modality at a different volume of tissue. Modulation may include up-regulating, down-regulating, blocking, etc. Modulation may include causing action potentials, blocking action potentials, or affecting the nerve by sub-action-potential processes. Modulation is thus used as a catch-all, and a specific mechanism of action is not to be inferred from this usage. Some examples below describe particular waveforms and/or effects which can fall into the broad category of modulation. The words “modulation” and “stimulation” may be used interchangeably herein.

In the context of a patient who has suffered from a trauma, such as a subarachnoid hemorrhage from stroke, aneurysm, or other vascular event affecting the brain, subsequent inflammatory response can be harmful. The inflammatory response may occur hours or days after the causative event. Additional discussion and details of underlying causes and effects of the post-stroke inflammatory response are in World Intellectual Property Organization Pub. No. WO2023059760, published Apr. 13, 2023, titled SYSTEMS AND METHODS FOR REDUCING INFLAMMATION IN THE CENTRAL NERVOUS SYSTEM, the disclosure of which is incorporated herein by reference.

There are numerous other injuries to the brain that can be associated with, cause, or lead to inflammation, and in many cases controlling or modulating that inflammation may aid in the healing process and/or prevent further injury. Concussions, for example, are (often mild) traumatic brain injuries caused by a blow to the head or sudden acceleration or deceleration of the head. Brain contusion, including cerebral contusion, is generally more severe than concussion, and involves actual bruising of the brain tissue. Cerebral or other intracranial hemorrhage involves bleeding in the brain, which can be caused by trauma or conditions such as aneurysms or strokes. Diffuse axonal injury involves damage to the brain's white matter caused by rapid acceleration or deceleration, leading to widespread axonal damage. Traumatic brain injury (TBI) relates to a broad category of brain injuries caused by external forces, ranging from mild concussions to severe injuries with long-term consequences. Penetrating head injury involves damage to the brain caused by a foreign object penetrating the skull, such as a bullet or a sharp object. Cerebral edema involves swelling of the brain tissue due to various causes, including trauma, infection, or stroke. Hypoxic-ischemic brain injury involves damage to the brain cells due to lack of oxygen or blood flow, leading to cell death and potential long-term deficits. Skull fracture, which involves a break in one or more of the bones of the skull can lead to brain injury, including, for example, if the fracture extends into the brain tissue. These various injuries may overlap and are neither exclusive nor exhaustive of the injuries that can affect the brain and lead to inflammatory response that may cause further damage and/or impair or delay the healing process. Treatment to reduce or modulate such inflammation may be desired.

In addition to injuries as the cause of inflammatory response, various disease conditions and/or infections can be associated with inflammation. For example, encephalitis is an inflammation of the brain usually caused by viral infections such as herpes simplex virus, West Nile virus, or autoimmune reactions. Meningitis is an inflammation of the protective membranes covering the brain and spinal cord, which can be caused by bacterial, viral, or fungal infections. Multiple Sclerosis (MS) is an autoimmune condition where the immune system attacks the protective covering of nerves in the brain and spinal cord, leading to inflammation and damage. A brain abscess is a collection of pus within the brain tissue, often caused by bacterial infections that lead to inflammation. Inflammation can occur around brain tumors as the body's immune response tries to contain or eliminate the abnormal growth. Autoimmune encephalitis includes inflammatory conditions caused by the immune system attacking healthy brain tissue, leading to symptoms such as seizures, cognitive impairment, and psychiatric symptoms. Neurodegenerative diseases and conditions, such as Alzheimer's disease, Parkinson's disease, and amyotrophic lateral sclerosis (ALS), can involve chronic inflammation in the brain, contributing to disease progression and symptoms. HIV-associated neurocognitive disorders (HAND) can also cause inflammation in the brain as a result of HIV infection, leading to cognitive impairment and other neurological symptoms. Treatment to reduce or modulate such inflammation may be desired.

Inflammation in the brain may also occur as a result of clinical conditions associated with bodily inflammation. Some illustrative conditions include, for example and without limitation, rheumatoid arthritis, osteoarthritis, inflammatory bowel disease (Crohn's disease, ulcerative colitis), psoriasis, asthma, chronic obstructive pulmonary disease (COPD), chronic sinusitis, chronic kidney disease, periodontal disease, diabetes, atherosclerosis, hepatitis, pancreatitis, endometriosis, and/or gout. Treatment to reduce or modulate such inflammation may be desired.

For patients suffering from any of these injuries, diseases, infections or conditions, the ability to control or modulate the inflammatory response may be helpful to recovery or healing or alleviation of other symptoms. Likewise, reducing, modulating or otherwise changing the degree of inflammation the patient experiences may prevent further injury in some circumstances.

Inflammation can also play a key role as patients are recovering from central or systemic insults in the intensive care unit (ICU). Inflammation can be associated with a variety of diagnoses, including for example and without limitation, sepsis, acute respiratory distress syndrome (ARDS), pneumonia, pancreatitis, peritonitis, meningitis, acute kidney injury, acute liver failure, acute myocardial infarction and congestive heart failure, traumatic brain injury, burns, surgical complications, systemic inflammatory response syndrome (SIRS), multi-organ dysfunction syndrome (MODS), and/or post-operative infections These conditions can lead to systemic inflammation, which can further exacerbate organ dysfunction and contribute to poor outcomes in critically ill patients. Close monitoring and prompt treatment of inflammation are crucial in managing patients in the ICU.

The aim in some examples herein is to provide a wearable device which is compact and non-intrusive, being easily placed and operated for a patient during an in-hospital stay for example in the intensive care unit. Some examples may have an intended life of up to two weeks, after which the device is intended to be discarded. Alternatively, the device can be discarded after each individual use and/or treatment. Other examples may provide such a wearable device, but for use at home or in other contexts and for different time durations. Some examples are characterized by a lack of external leads or lead wires, such that therapy electrodes and anchoring devices are all in a single housing, which may include a clip or may be used with adhesive tape for securing the apparatus in place.

The ICU is one example of a potential care environment. Others may include, for example and without limitation, hospitals generally, rehabilitation centers, pain management clinics, physical therapy clinics, neurology clinics, sports medicine clinics, chiropractic clinics, home health care settings, long-term care facilities including residential care or assisted living facilities, outpatient surgery centers, urgent care facilities, emergency rooms, convalescent homes, hospice centers, birthing centers, or any other medical care location or facility. The system and/or device may be provided as well for use in ambulances or other use cases, such as a first aid tent at a sporting event, outside of the controlled environment of a hospital or other care facility.

A range of therapy products, including certain drugs, gene therapies, antibodies and/or antibiotics, and cellular therapies offer promise of clinical benefits. Several such therapies have shown a propensity to inflammatory side effects. It should be noted that the following examples include drugs that can cause inflammatory side effects having benefits that often outweigh the risks for many patients. Proper monitoring and management of side effects are crucial aspects of treatment with these medications.

Statins are a class of drugs widely prescribed for lowering cholesterol. Some patients experience a paradoxical side effect in the form of increase inflammation. The drug class includes, for example and without limitation, Atorvastatin, Simvastatin and/or Rosuvastatin. Reported inflammatory side effects have included myalgia and myositis, each of which are forms of inflammation in muscle tissue that lead to pain and weakness. More rarely observed is the inflammatory side effect of rhabdomyolysis, which can cause severe muscle breakdown due to inflammation. Some patients also demonstrate increased C-reactive protein (CRP) levels, which is a known marker for inflammation. It is believed that the mechanism of action for the inflammatory side effects of statins may include activation of the NLRP3 inflammasome, leading to the production of pro-inflammatory cytokines, such as IL-1β.

Chemotherapy agents can cause inflammation as a side effect of their cytotoxic actions. Inflammatory side effects may occur, without attempting to provide a comprehensive list here, in association with any of Doxorubicin (Adriamycin), Bleomycin, and/or 5-Fluorouracil (5-FU). The inflammatory side effects have been observed in various forms, including mucositis, an inflammation of the mucous membranes lining the digestive tract, pneumonitis, an inflammation in the lungs, cardiotoxicity, an inflammation of heart muscle tissue, skin inflammation, and/or hand-foot syndrome. It is believed that the mechanism of action for the inflammatory side effects of chemotherapy agents may include increased oxidative stress and triggered release of pro-inflammatory cytokines such as TNF-α, IL-1β, and IL-6.

Immune checkpoint inhibitors may also cause inflammatory side effects. While these drugs are designed to enhance immune responses against cancer, inflammatory side effects have been observed. Illustrative and non-limiting examples include Pembrolizumab (Keytruda), Nivolumab (Opdivo), and/or Ipilimumab (Yervoy), though this list is not intended to be limiting. Specific inflammatory side effects that have been observed include colitis, an inflammation of the colon, pneumonitis, hepatitis (liver inflammation), and/or endocrinopathies, which is an inflammation affecting endocrine glands. Dermatitis (skin inflammation and rashes), myocarditis (heart muscle inflammation, which is rare but potentially severe, as well as neurological effects (encephalitis, peripheral neuropathy) can also arise. The mechanism of action here is believed to be that these pro-immune response drugs remove the “brakes” on the immune system, and can thereby lead to overactivation of T cells, resulting in inflammatory responses in various organs. To identify such side effects, regular monitoring of organ function and inflammatory markers can be performed, to allow prompt recognition and grading of immune-related adverse events (irAEs). Treatments can include the use of corticosteroids for moderate to severe irAEs, potential use of other immunosuppressive agents in steroid-refractory cases, and/or temporary or permanent discontinuation of checkpoint inhibitor therapy in severe cases.

Some antibiotics can cause inflammation, either directly or by disrupting the gut microbiome. Non-limiting examples include fluoroquinolones, sulfonamides, and/or, at least in allergic persons, penicillins. Inflammatory side effects can include, for example, tendinitis and tendon rupture (particularly with fluoroquinolones), Stevens-Johnson syndrome (a severe skin inflammation), and/or antibiotic-associated colitis. As for mechanism of action, fluoroquinolones can directly stimulate pro-inflammatory cytokine production, and others may disrupt the gut microbiome, leading to dysbiosis and inflammation.

While primarily anti-inflammatory, long-term use of NSAIDs can paradoxically increase inflammation in some tissues. NSAIDs may include, for example, Ibuprofen, Naproxen, and Diclofenac. The inflammatory side effects may include, without limitation, gastritis and peptic ulcers, increased intestinal permeability leading to low-grade systemic inflammation, and, rarely, aseptic meningitis. The mechanism of action may be that chronic NSAID use can disrupt the gut mucosal barrier, leading to increased translocation of bacteria and bacterial products, triggering systemic inflammation.

Stevens-Johnson syndrome can also be observed as a rare and severe reaction to ibuprofen and other medicines, including allopurinol in relatively higher doses, in acute and/or chronic usage.

Some hormonal treatments, particularly in cancer therapy, can lead to inflammatory side effects. Illustrative and non-limiting examples include Tamoxifen (breast cancer), and Leuprolide (prostate cancer). With tamoxifen, the side effect may manifest as increased risk of endometrial inflammation and rarely, endometrial cancer. Leuprolide can cause a temporary increase in testosterone, leading to “tumor flare” and associated inflammation. These drugs can alter hormonal balances, which in turn can affect inflammatory pathways and immune cell function.

Some antipsychotic medications, particularly atypical antipsychotics, can induce low-grade inflammation. Illustrative and non-limiting examples include clozapine, olanzapine, and risperidone. The inflammatory side effects may manifest as metabolic syndrome, which is associated with chronic low-grade inflammation, weight gain and insulin resistance, which can promote inflammation, and, rarely, myocarditis, an inflammation of cardiac tissue. The mechanisms of action here may be that the drugs can affect metabolic pathways and adipose tissue function, leading to increased production of pro-inflammatory adipokines.

Some medications used to treat Alzheimer's disease can have inflammatory side effects, particularly those targeting amyloid plaques. Examples include aducanumab, lecanemab, and/or donanemab, which has been approved for use in some but not all geographies. The observed inflammatory side effects may include Amyloid-Related Imaging Abnormalities (ARIA), such as ARIA-E (edema) and/or ARIA-H (microhemorrhages and hemosiderosis), as well as other brain swelling and/or microbleeds in the brain. These drugs, particularly monoclonal antibodies targeting amyloid-beta, can cause localized inflammation in the brain as they clear amyloid plaques. This inflammatory response can lead to fluid accumulation (edema) and small bleeds in some patients. For example, ARIA-E (edema) is thought to be due to increased vascular permeability caused by the inflammatory response to rapid amyloid clearance. ARIA-H (microhemorrhages) may occur due to weakening of blood vessel walls as amyloid plaques are removed, combined with local inflammatory processes. Regular MRI scans are typically required to monitor for ARIA complications, and in some cases, treatment may need to be paused or discontinued if severe ARIA occurs. Anti-inflammatory treatments may be considered to manage symptoms in some cases. It is important to note that while these inflammatory side effects can be serious, they are often manageable with close monitoring. The potential benefits of these drugs in slowing cognitive decline must be weighed against the risks for each individual patient.

Chimeric Antigen Receptor T-cell (CAR-T) therapies are a form of immunotherapy used primarily in certain blood cancers. They can cause significant inflammatory side effects. Illustrative drugs may include tisagenlecleucel (Kymriah), axicabtagene ciloleucel (Yescarta), and Brexucabtagene autoleucel (Tecartus). Inflammatory side effects can include Cytokine Release Syndrome (CRS), which can lead to fever, hypotension, hypoxia and, in severe cases, organ dysfunction, coagulopathy, Immune Effector Cell-Associated Neurotoxicity Syndrome (ICANS), which can cause confusion, delirium, aphasia, seizures and, in severe cases: cerebral edema, Macrophage Activation Syndrome (MAS)/Hemophagocytic Lymphohistiocytosis (HLH), which can lead to persistent fever, cytopenias, liver dysfunction, hyperferritinemia, and/or coagulopathy, and B-cell aplasia, which causes limits antibody production and leads to increased risk of infections. The mechanism of action is that CAR-T cells, once infused, recognize and attack cancer cells. This massive cellular destruction leads to the release of large amounts of cytokines (e.g., IL-6, IFN-γ, TNF-α), causing systemic inflammation. The rapid expansion of CAR-T cells and their cytokine production can also affect the central nervous system, leading to neurotoxicity. Management is performed by close monitoring in specialized centers, supportive care for mild CRS, Tocilizumab (IL-6 receptor antagonist) for moderate to severe CRS, corticosteroids for severe CRS or ICANS, ICU care for severe cases, and potential use of newer cytokine-directed therapies (e.g., anakinra, siltuximab) in refractory cases.

While many diabetes drugs aim to reduce inflammation associated with the disease, some can have pro-inflammatory effects or side effects related to inflammation. Examples include the class of thiazolidinediones (TZDs), including for example, pioglitazone (Actos) and rosiglitazone (Avandia). These drugs are generally anti-inflammatory, but can cause fluid retention leading to edema, and in rare cases, drug-induced liver injury with inflammatory component. The mechanism of action is that TZDs are PPAR-γ agonists that generally reduce inflammation, however, they can cause fluid retention, which may be associated with a mild inflammatory response in some tissues. Another class of diabetes drugs are the SGLT2 Inhibitors, including empagliflozin (Jardiance), dapagliflozin (Farxiga), and canagliflozin (Invokana). The SGLT2 inhibitors can cause an increased risk of genital mycotic infections and urinary tract infections, and rare cases of Fournier's gangrene (necrotizing fasciitis of the perineum). SGLT2 inhibitors may cause an inflammatory side effect by increasing glucose excretion in urine, creating an environment conducive to infections, which trigger inflammatory responses. Another class of diabetes drugs includes DPP-4 Inhibitors, such as sitagliptin (Januvia) and saxagliptin (Onglyza). Again, while the DPP-4 Inhibitors are generally considered anti-inflammatory, these can be associated with rare cases of drug-induced pancreatitis by mechanisms that are not yet fully understood. Additional diabetes drugs include sulfonylureas, such as glipizide (Glucotrol) and glyburide (Diabeta), which can indirectly promote inflammation through weight gain and hypoglycemia. Weight gain can increase overall inflammation in the body. Hypoglycemic episodes can trigger stress responses that include inflammatory components. The diabetes drug metformin is also generally considered anti-inflammatory, but can cause rare cases of drug-induced liver injury with inflammatory component through idiosyncratic reactions. Another class of diabetes drugs, some of which have become popular for use as general weight loss drugs, are the GLP-1 Receptor Agonists, including liraglutide (Victoza) and semaglutide (Ozempic, Rybelsus), which are also associated with rare cases of pancreatic inflammation through mechanisms not fully elucidated.

For each of these drugs used for treatment of diabetes, regular monitoring of organ function, particularly liver and pancreas is performed. Prompt recognition and management of infections is also important, and careful titration of doses to minimize risk of hypoglycemia. Patients are often educated on recognizing signs of rare but serious side effects. For patients with history of pancreatitis, alternative therapy may be carefully considered.

Vagal nerve stimulation (VNS), and its impact on inflammation is a fascinating area of research that has gained significant attention in recent years. This approach leverages the complex interactions between the nervous system and the immune system to modulate inflammatory responses.

The vagus nerve, also known as the 10th cranial nerve, is a key component of the parasympathetic nervous system. It plays a crucial role in the “inflammatory reflex,” a neural circuit that regulates immune responses and inflammation. This reflex consists of the afferent (sensory) arm, which detects inflammatory mediators and signals to the brain, and the efferent (motor) arm, which sends anti-inflammatory signals from the brain to the periphery.

VNS primarily impacts inflammation through the cholinergic anti-inflammatory pathway. Stimulation of the vagus nerve leads to the release of acetylcholine in organs of the reticuloendothelial system (e.g., spleen, liver). Acetylcholine binds to α7 nicotinic acetylcholine receptors (α7nAChR) on macrophages and other immune cells, and this binding inhibits the production of pro-inflammatory cytokines, particularly tumor necrosis factor (TNF), interleukin-1β (IL-1β), and interleukin-6 (IL-6).

There are also spleen mediated effects of VNS. Effective VNS activates the splenic nerve, which releases norepinephrine. In turn, norepinephrine interacts with beta-adrenergic receptors on T cells in the spleen. These stimulated T cells release acetylcholine, which then acts on nearby macrophages to suppress inflammation.

VNS can directly modulate the function of various immune cells. For example, VNS reduces neutrophil recruitment and activation. Also, as to macrophages, VNS promotes a shift from pro-inflammatory M1 to anti-inflammatory M2 phenotype. VNS also modulates T cell differentiation, potentially favoring regulatory T cells.

VNS may also induce systemic anti-inflammatory effects by, for example, reducing circulating levels of pro-inflammatory cytokines. VNS can also decrease activation of the hypothalamic-pituitary-adrenal (HPA) axis, and attenuates the acute phase response, including C-reactive protein production.

Recent research suggests that VNS can inhibit the NLRP3 inflammasome, a key mediator of inflammatory responses. VNS may, for example, decrease assembly and activation of the NLRP3 inflammasome complex, and/or reduce production of IL-1β and IL-18, which are processed by the inflammasome.

With these general effects, VNS has shown promise in various inflammatory disorders. For example, with Rheumatoid Arthritis, VNS may reduce joint inflammation and pain. With Inflammatory Bowel Disease, VNS can improve mucosal healing and reduce pro-inflammatory cytokines. VNS in response to sepsis can attenuate the systemic inflammatory response and improves survival in animal models. VNS has also shown potential for reducing inflammation in conditions like stroke and traumatic brain injury.

It is generally believed, but without limiting the present invention to these specific considerations, that parameters of VNS can influence its anti-inflammatory effects. For example, it has been observed in some cases that lower frequencies (1-10 Hz) are generally more effective for anti-inflammatory effects. Moderate intensities often provide optimal balance between efficacy and side effects. Both acute and chronic stimulation paradigms have shown benefits, with some effects persisting after stimulation ceases.

In view of the above, VNS represents a promising approach in the emerging field of bioelectronic medicine. VNS offers a potential alternative or adjunct to pharmacological anti-inflammatory therapies, and may provide more targeted regulation of inflammation with fewer systemic side effects. Ongoing research aims to develop less invasive methods of VNS, such as transcutaneous approaches.

As an example, a clinical study of Subarachnoid hemorrhage (SAH) is now described. SAH resulting from a ruptured aneurysm accounts for 7% of all strokes worldwide, with reports indicated up to 40% of patients suffer permanent disability. Secondary injury is a major driver of morbidity following SAH, as mediated by early brain injury, cerebral vasospasm, delayed cortical ischemia, and chronic hydrocephalus.

Inflammation is thought to be a key factor driving the morbidity associated with SAH. Following SAH, blood within the subarachnoid space triggers local and systemic inflammatory responses with increases in the inflammatory markers IL-6 and TNF-α both systemically and centrally having been reported. These increased inflammatory markers are correlated with adverse sequelae from SAH, including the risk for vasospasm, cerebral edema, hydrocephalus, and poor overall patient outcome.

Despite increasing evidence for the role of inflammation following SAH, an effective method to modulate the deleterious inflammatory response in patients following SAH is lacking. Pharmacologic approaches that target anti-inflammatory pathways have been unsuccessful in clinical trials. VNS provides a novel, non-pharmacologic approach to systemic immunomodulation. VNS reduces inflammation via a cholinergic anti-inflammatory pathway, whereby efferents from the vagus nerve to the spleen and other organs leads to the release of acetylcholine, with downstream inhibition of cytokine release in macrophages. Studies have demonstrated that VNS reduces systemic inflammatory markers and has had early success treating inflammatory conditions such as arthritis, sepsis, and inflammatory bowel disease. Early evidence has also emerged that non-invasive approaches via transcutaneous auricular vagus nerve stimulation (taVNS) can accomplish similar effects. Thus, a clinical study hypothesis was that implementing ta VNS in the acute period following spontaneous SAH would attenuate the expected inflammatory response to hemorrhage and curtail clinical morbidity.

The Non-invasive Auricular Vagus nerve stimulation for Subarachnoid Hemorrhage (NAVSaH) randomized control trial was directed to this subject matter. The primary aims of this trial were to determine if ta VNS following SAH reduces TNF-α in the plasma and CSF and reduces the rate of radiographic vasospasm. Exploratory analyses also evaluated the change in clinical outcomes of patients via blinded assessment with modified Rankin Scale scores (mRS) at discharge and follow-up. Further exploratory studies were to examine if taVNS alters glycemic control. In aggregate, the findings support taVNS as a potential novel modality to treat SAH-induced inflammation and associated clinical sequelae.

The NAVSaH study was a prospective, triple-blind, randomized controlled trial with two study arms, with assessment based on intention to treat with regard to the assigned treatment arm. Participants were persons having an acute, spontaneous (non-traumatic) SAH. All candidates were screened based on inclusion and exclusion criteria, consent was obtained, and subjects were enrolled within 24 hours of presentation with SAH. Patients were randomized to receive either taVNS or sham treatment at a 1:1 ratio in a blinded study.

Following enrollment and randomization into a treatment arm, initial samples of blood and CSF (if the patient had an external ventricular drain in place) were collected. The patient began either taVNS or sham stimulation 20 minutes twice daily, with an AM session between 05:00-10:00 and a PM session between 16:00-21:00. Treatment sessions occurred twice a day throughout a patient's intensive care unit stay, and therefore the cumulative number of days of therapy varied by patient, with all patients receiving at least 7 days of therapy. All patients were fitted with a portable TENS (transcutaneous electrical nerve stimulation) unit connected to two ear clips applied to the left ear during treatment periods. For VNS treatment, these ear clips were placed along the concha of the ear; for sham treatments, the clips were placed along the ear lobe to avoid stimulation of the auricular vagus nerve from tactile pressure alone in the absence of current.

Referring to FIG. 1B, the location of taVNS and sham electrodes are illustrated. For the study, inclusion criteria included an age over 18, confirmed aneurysm, and acceptable patient or family informed consent. Exclusion criteria included immune-suppression cancer therapy, presence of an active implantable medical device (pacemaker, neurostimulator, etc.), and bradycardia at admission. The included patients were then randomized between taVNS and sham arms. Treatment schedules included AM and PM treatments for up to 13 days, with collection of blood and cerebrospinal fluid (CSF) samples on days 1, 4, 7, 10 and 13 or until release from the ICU, whichever came first.

Stimulation parameters were selected based on prior studies that sought to maximize vagus nerve stimulation while avoiding the perception of pain. Stimulation parameters used in this trial for the ta VNS arm were a duration of 20 minutes, frequency of 20 Hz, pulse width of 250 μs, and an intensity of 0.4 mA. Sham treatments involved no electrical current, and also had a 20-minute duration. The patient cohorts for Sham and taVNS arms were similar according to both Hunt Hess scores, and Fisher Grade.

One primary outcome assessed was the change in the key inflammatory marker TNF-α in the plasma and CSF in SAH patients undergoing the taVNS versus sham treatment. IL-6 was also evaluated as a secondary outcome in an exploratory manner. Blood samples and CSF samples, when a ventriculostomy was in place, were collected every three days throughout patient hospitalization following acute SAH, as noted above. Baseline blood and CSF samples were collected prior to the first treatment session. Blood and CSF samples were collected and processed immediately.

Another primary outcome assessed was the difference in the presence of moderate or severe radiographic vasospasm between the taVNS and sham stimulation groups, as assessed by blinded clinician assessment and quantitatively via measurement of vessel caliber on serial exams. In addition to initial diagnostic testing, all patients underwent a repeat CT or catheter angiogram approximately seven days after admission per the institution's protocol. Further vascular imaging was also performed if there was a clinical concern for clinical vasospasm or stroke. For both planned and indicated imaging sessions, each vascular imaging study was reviewed by a neuroradiologist or endovascular neuro-interventionalist blinded to treatment arm, who described the overall imaging study as it related to vasospasm as none, mild, moderate, or severe to account for proximal vessel caliber and overall/distal perfusion.

To assess radiographic vasospasm more quantitatively, all radiological images were reviewed by a single physician reviewer blinded to patient identity, cohort assignment, and indication for the imaging study. The reviewer was fellowship-trained in neurointerventional radiology. Images were in DICOM format and reviewed and measured with the RadAnt™ DICOM viewer software. Cerebral vessel diameters were measured bilaterally at specific locations on maximally magnified images, with consistent measurement locations maintained across serial imaging studies. Vessel diameter on each serial image was normalized to the initial angiographic test, with comparisons made only between matching modalities and accounting for anatomical variations or imaging limitations. Vasospasm was defined for each vessel compared with baseline as none/mild if <25%, moderate if 25-50%, and severe if >50%. Vessel caliber was also analyzed over time by treatment group.

Referring to FIG. 1C, the results in terms of vasospasm are shown, with the treatment group shown to the right of the control group with bar graphs for each of any radiographic vasospasm, and moderate or severe vasospasm. Improvement as a percentage of subjects experiencing vasospasm is clearly shown.

Secondary outcomes also included the number of vascular imaging studies obtained and interventions undertaken to treat vasospasm, including 1) blood pressure augmentation while in the intensive care unit, 2) treatments performed during catheter angiogram, such as administration of intraarterial vasodilators, 3) the use of intrathecal vasodilators. The presence of infarct on subsequent CT or MRI was also recorded, as a surrogate marker for delayed cerebral ischemia.

Additional secondary outcomes included clinical outcome metrics, including discharge destination (home, inpatient rehabilitation facility, skilled nursing facility, hospice, or death), and mRS scores at discharge and first follow-up.

The estimated required sample size for the pilot NAVSaH clinical trial was 50 patients. However, enrollment to study this endpoint was terminated early following an interim analysis, as the effect size for the reported primary aim (presence of moderate/severe radiographic vasospasm) was larger than predicted. In the initial trial design based on early preliminary data, there was anticipated to be a 30% reduction in moderate/severe vasospasm in the taVNS treated arm, but interim analysis demonstrated a >40% reduction in the treatment arm. Therefore, results are reported based on 27 patients who completed their initial hospital stay and at least their first outpatient follow-up. In other words, the study was terminated early as the results were unexpectedly favorable to the taVNS therapy performing more effectively than initially modeled when the study size was planned.

Patient demographics and clinical characteristics were summarized using counts and frequencies for categorical variables or means and standard deviations for continuous variables. The distributions of baseline patient characteristics across taVNS and Sham stimulation groups were compared using Student t-test or Chi-square test as appropriate. Between-group differences in the cross-sectional outcomes (such as the presence of moderate or severe radiographic vasospasm, discharge destination, etc.) were compared using a t-test or Chi-square test as appropriate. Between-group differences in these repeatedly measured outcomes (such as modified Rankin Scale (mRS, coded as mRS<3 or not), biomarkers of cytokines, etc.) were compared using linear mixed models (for normality data) or generalized linear mixed models (for non-normality data) to account for potential correlation among multiple measures taken from the same patients. The fixed effects of the model included intervention groups (taVNS vs. Sham), the measurement times (treated as a categorical variable), and their interaction. The random effect included subject-specific intercept which described the average deviation of an individual from the overall level at baseline. One-sided post-hoc tests were performed to assess whether taVNS improved outcomes at specific time points. Normalized vessel caliber was also assessed using a similar mixed model to estimate its average change over time and to compare effect of taVNS on vasospasm, where the measurement time was treated as a continuous variable instead. The assumption of normality was assessed graphically based on residuals out of models. All the analyses were performed using SAS 9.4 (SAS Institutes, Cary, NC). Unless stated otherwise, all the statistical tests were one-sided for outcome variables and two-tailed for baseline characteristics, with a p-value of <0.05 for significance.

Sixty-four patients were assessed for eligibility for enrollment in the trial between January 2021 and October 2023. Of those, 27 met inclusion criteria and were randomized to a treatment arm (13 taVNS and 14 patients sham). One patient in each study arm ceased treatment sessions and laboratory draws before the completion of the intervention. 27 patients were analyzed for clinical outcomes based on intention-to-treat. 26 patients were analyzed for laboratory inflammatory cytokine endpoints, with one patient in the taVNS group excluded from analysis since only a single baseline lab draw was performed.

Patients in both treatment arms were not significantly different with regard to gender, race, or method of aneurysm treatment. They were also similar with regards to Glasgow Coma Scale (GCS), Hunt & Hess Score, and modified Fisher Scale Score on admission. There were no reportable adverse events, including no reports of pain or irritation related to the stimulation site.

When interpreted by blinded evaluators, patients treated with taVNS had a significant reduction in the presence of any radiographic vasospasm (p=0.035), or radiographic vasospasm described as moderate or severe (p=0.018), both illustrated in the graphics of FIG. 1C. When cerebral vessel diameter on serial vascular studies were measured, there was also a significant reduction in the number of vessels with moderate or severe vasospasm when normalized to baseline (p=0.015) in the taVNS treated group.

FIG. 1D shows that the normalized vessel calibers significantly increased over time in the ta VNS group (with average daily change=0.030, 95% CI=0.012-0.047), while the average daily change in the sham group is −0.002 (95% CI=[−0.013, 0.008]). The serial vascular studies with normalized vessel caliber showed a significant interaction effect between day and treatment (p=0.0026).

The mean number of follow up vascular studies obtained was 2.8 (standard deviation=0.8) for the taVNS group and 3.4 (standard deviation=1.2) for the sham group. There were numerically fewer interventions performed for vasospasm, including blood pressure augmentation, intraarterial vasodilator infusion or angioplasty during angiography, and intrathecal vasodilator administration, with no significant differences between groups in this initial cohort.

FIG. 1E shows several comparisons of results for plasma and CSF constituents of interest, with the treatment arm represented by broken lines in each panel, and the sham arm by solid line in each of the four panels. A total of 26 patients were included in the analysis of plasma TNF-α and IL-6, with one patient excluded due to only having a single baseline level collected. The pro-inflammatory cytokine TNF-α was significantly reduced in the plasma on treatment days 7 and 10 in patients treated with taVNS (p=0.015 and p=0.030, respectively) (FIG. 1E, upper right). The plasma IL-6 was significantly lower on day 4 in patients treated with taVNS (p=0.024) (FIG. 1E, upper left). A total of 13 patients had an extra-ventricular drain in place (n=6 taVNS, n=7 sham) and were included in the analysis of CSF TNF-α and IL-6. The pro-inflammatory cytokines TNF-α and IL-6 were both significantly reduced in the CSF on treatment day 13 in patients treated with taVNS (p=0.031 and p=0.025, respectively) (FIG. 1E, lower panels).

Blinded assessments of the mRS scores were performed at admission, discharge, and at first outpatient follow-up. One patient in each of the taVNS and sham groups was lost to follow-up, with no mRS assessed after discharge. mRS scores on admission were not significantly different between groups. A good outcome, as assessed via mRS, refers to scores 0-2, while a poor outcome refers to scores 3-6. The change in mRS over the course from admission to first follow-up for all patients is illustrated in FIG. 1F, with sham shown in solid line, and treatment arm in a broken line.

Patients receiving taVNS demonstrated higher rates of favorable outcomes compared to those in the sham group both at discharge (38.4% vs 21.4%) and at first follow-up (76.9% vs 57.1%), with a significant difference in improvement observed in the taVNS group from admission to first follow-up (p=0.014), while no significant difference was noted in the sham group (p=0.18). Following hospitalization for SAH, patients had one of 5 discharge outcomes: home, inpatient rehabilitation facility, skilled nursing facility, hospice, or death. No patient in either treatment arm died before discharge from the hospital. With discharge destinations divided into “good” discharge (home and inpatient rehabilitation) versus “poor” discharge (skilled nursing facility, hospice, or death), there was a significantly lower rate of poor discharge in patients treated with taVNS (p=0.04).

This study is the first to use non-invasive neuromodulation to mitigate inflammation in SAH patients. Despite improvements in managing SAH patients, mortality and morbidity remain high. Targeting post-hemorrhage inflammation is important for improving outcomes. The study showed that ta VNS can significantly reduce key inflammatory cytokines (TNF-α and IL-6), reduce cerebral vasospasm, and improve clinical outcomes. These findings provide a new therapeutic approach for treating SAH sequelae.

Following SAH, blood within the subarachnoid space triggers central and systemic inflammatory responses. Key drivers of SAH-induced inflammation are the cytokines TNF-α and IL-6. In animal models and humans, these cytokines are associated with vasospasm, hydrocephalus, delayed cerebral ischemia, and poor outcomes. The relationship between increased inflammation and worsened clinical outcomes is felt to arise from several mechanisms that include inducing vasoconstriction, degrading the blood brain barrier, and prompting neuronal cell death within the parenchyma itself.

Numerous anti-inflammatory interventions have been trialed in humans following SAH to target inflammatory pathways. In smaller enrollment studies, there has been some early evidence of clinical benefit with Cyclosporine A and steroids. Other medications, such as Clazosentan, Cilostazol, and IL-1 antagonists demonstrated no impact on overall outcomes. In larger meta-analysis studies, Simvastatin, Aspirin, non-steroidal anti-inflammatory medications, and thienopyrindines all demonstrated no improvement. Thus, while some pathway-targeted pharmacological approaches have led to changes in secondary outcomes of vasospasm and delayed cerebral ischemia in clinical trials, these approaches have ultimately failed to produce an effective intervention that reliably improves functional or neurological outcomes in SAH patients. This dissociation between early trial endpoints and a lack of improved neurological clinical outcomes suggests that a narrow, molecular pathway-oriented approach may be insufficient to address the broad cascade of physiologic drivers that result in poor clinical outcomes following SAH.

VNS may provide a broader immunomodulatory approach. VNS has been postulated to reduce inflammation through the cholinergic anti-inflammatory pathway. Sensory neurons activated by infection or injury travel to the brainstem via the vagus nerve. Subsequent neural efferent input to the spleen and other organs leads to the release of acetylcholine, which interacts with α7 nicotinic acetylcholine receptors on immunocompetent cells, inhibiting cytokine release in macrophages. VNS has been successfully used in models of cerebral ischemia, reperfusion, rheumatoid arthritis, sepsis, inflammatory bowel diseases, and cerebral aneurysms and SAH. Clinically, VNS has historically been performed by surgical cervical neck dissection and placement of a cuff electrode directly around the nerve. Alternatively, as disclosed herein, VNS can be accomplished non-invasively by stimulating the auricular branch of the vagus nerve as it courses through the external ear. This non-invasive and low-risk form factor lends itself to deployment in ICU patients who are medically fragile and would not tolerate surgical implantation of a device. Prior to this study, however, there has been a dearth of understanding of VNS effects on SAH patients.

This prospective, randomized trial is the first to report the impact of non-invasive VNS applied following spontaneous SAH. This auricular approach to VNS demonstrates a significant reduction in radiographic vasospasm as assessed by two key metrics: assessment of overall clinical vasospasm by blinded radiology interpreters and quantitative serial vessel caliber measurements. The blinded assessor's interpretation of vasospasm captures the qualitative radiologic assessment that accounts for large proximal vessel caliber changes, as well as harder-to-quantify distal perfusion alterations from medium vessel caliber changes. The serial quantitative analysis more analytically compares changes in the large proximal vessels over time, limiting potential differences in qualitative assessments by different neuroradiologists. The finding of significant changes in radiographic vasospasm is a substantial finding, but alteration in radiographic vasospasm alone has not historically been directly or clearly related to changes in functional outcome. Thus, the significant reduction in “poor” discharge destinations (skilled nursing facilities and hospice) and early evidence of superior improvement in mRS between admission and first follow-up suggests that the impact of taVNS is not limited to improvement in radiographic vasospasm alone. Despite the promising nature of these preliminary findings, further validation and study of clinical outcomes in a larger cohort will be needed. For example, the outcomes included numerically fewer patients assigned a mRS of 0 in the taVNS group at first follow-up compared to the sham group (1 vs 3, respectively), despite an improvement in the average mRS in the taVNS group.

The hypothesized mechanism for this vascular and clinical effect was due to taVNS's ability to blunt the deleterious inflammatory response following SAH. Therapy using taVNS can achieve significant reductions in TNF-α and IL-6 both centrally and systemically. Notably, these effects seem to have different time scales. While serum markers generally showed a more linear trend of reduced TNF-α and IL-6 from the onset of treatment, the difference in CSF TNF-α and IL-6 was more delayed and significantly different at day 14. These findings suggest that the serial interventions (i.e., 20 min, twice daily, for 14 days) may have a cumulative effect over time to achieve the full clinical benefit. While these findings validated the immunomodulatory impact of taVNS, a more comprehensive evaluation of inflammatory cytokines will be required in the future to define further how taVNS fully impacts the inflammatory cascade and the interplay of central and systemic inflammation.

While inflammation is being altered with taVNS, there may be other mechanisms that play a role. Following SAH, there is a widespread sympathetic response, which can manifest with increased risk for cerebral vasospasm, delayed cortical ischemia, and extracerebral organ damage. While no acute bedside perceptible alterations in cardiovascular metrics were seen in any of our study patients, our rigorous analyses of continuous heart rate, blood pressure, and respiratory data throughout patients' intensive care unit stays indicate treatment with VNS may restore the SAH-induced autonomic imbalance by enhancing parasympathetic input. Another phenomenon observed in SAH patients is cortical spreading depolarizations (CSDs), with evidence of a causal relationship between CSDs and worse neurological outcomes. VNS has been demonstrated to decrease CSDs in a study of migraine, which supports the possibility that taVNS following SAH may have a similar effect. These multi-pathway mechanisms of taVNS may explain the robust clinical benefits seen in this study, while narrowly focused pharmacologic approaches have thus far been unsuccessful.

The results of this study are encouraging, but the current report has limitations. The difference in electrode placement between treatment arms could allow detection of a group difference, although to which group a patient was assigned would not be clear. Clinician presumption of treatment arm could influence their decisions regarding other medical care or interventions. While this study was randomized, it was done at a single institution, and thus, one cannot infer yet whether these findings are more generalizable. This randomized cohort, while having a positive and robust effect, is still small. Importantly, the SAH population can be heterogenous and a patient's hospital course can be influenced by many factors. Therefore, in a small cohort there may be confounding factors including a patient's medical history, intraprocedural events during aneurysm treatment, or variability in management between clinicians that may influence patient outcomes. Ultimately, a larger multi-center clinical trial will be essential to fully validate the clinical effect of ta VNS and demonstrate that the treatment can be widely and effectively applied.

As illustrated in the study, taVNS may be used to mitigate inflammation in response to an event, here the SAH event experienced by the study patients. The present innovation relates to identifying taVNS as having potential effects to prevent or mitigate inflammation in response to a patient receiving one or more of the drugs that have been described previously. That is, the present inventors have inferred that ta VNS may be a non-invasive method to mitigate inflammatory-driven side effects for various drug therapies. By stimulating the auricular branch of the vagus nerve, taVNS can activate the cholinergic anti-inflammatory pathway, leading to a reduction in pro-inflammatory cytokines like IL-6 and TNF-α. This approach could potentially be used alongside drug therapies to reduce inflammation-related side effects in several contexts, discussed in the following paragraphs:

Statins, while effective for lowering cholesterol, can paradoxically increase inflammation in some cases. taVNS could potentially help mitigate side effects. For example, as to myalgia and myositis, by reducing IL-6 and TNF-α levels, taVNS may decrease muscle inflammation and associated pain. For Rhabdomyolysis, in severe cases, lowering systemic inflammation might help prevent or reduce the severity of muscle breakdown. For elevated C-reactive protein (CRP) levels, taVNS could help lower CRP, a marker of systemic inflammation. Statins are often taken chronically. taVNS may be used prophylactically by statin patients, such as with a once or twice daily regimen of 5-30 minutes of therapy, or more or less frequently. Episodic use of taVNS may be implemented in response to symptom onset or identification instead, with usage of taVNS in a once or twice daily regimen of 5-30 minutes of therapy for a period of days to weeks, or until cessation of symptoms.

Many chemotherapy agents cause inflammation as a side effect. taVNS could potentially help with several of the resulting side effects. For mucositis, reducing IL-6 and TNF-α levels may decrease inflammation of the mucous membranes lining the digestive tract. With pneumonitis, lowering systemic inflammation could help reduce lung inflammation. As for cardiotoxicity, decreasing inflammatory markers might help protect heart muscle from inflammation-induced damage. For hand-foot syndrome, reducing overall inflammation may help alleviate this painful skin condition. taVNS may be used prophylactically by some patients, such as with a once or twice daily regimen of 5-30 minutes of therapy, or more or less frequently. Episodic use of ta VNS may be implemented in response to symptom onset or identification instead, with usage of taVNS in a once or twice daily regimen of 5-30 minutes of therapy for a period of days to weeks, or until cessation of symptoms. In other examples, taVNS may be provided when chemotherapy is begun and/or changed, such as using taVNS for one to two weeks prophylactically.

Immune Checkpoint Inhibitors can lead to significant inflammatory side effects. taVNS could potentially help manage, for example, colitis by reducing IL-6 and TNF-α which in turn may decrease colon inflammation. For Pneumonitis, lowering systemic inflammation could help mitigate lung inflammation. For hepatitis, decreasing inflammatory markers may help reduce liver inflammation. For endocrinopathies, lowering overall inflammation might help reduce inflammation affecting endocrine glands. For dermatitis, reducing systemic inflammation could help alleviate skin inflammation and rashes. For myocarditis, decreasing inflammatory markers might help protect against heart muscle inflammation. taVNS may be used prophylactically by some patients, such as with a once or twice daily regimen of 5-30 minutes of therapy, or more or less frequently. Episodic use of taVNS may be implemented in response to symptom onset or identification instead, with usage of taVNS in a once or twice daily regimen of 5-30 minutes of therapy for a period of days to weeks, or until cessation of symptoms. In other examples, taVNS may be provided when immune checkpoint inhibitor therapy is begun and/or changed, such as using ta VNS for two weeks prophylactically when the therapy begins.

Some antibiotics can cause inflammation directly or by disrupting the gut microbiome. taVNS could potentially help with tendinitis and tendon rupture: Reducing IL-6 and TNF-α levels may help decrease inflammation in tendons, particularly with fluoroquinolones. For Stevens-Johnson syndrome: Lowering systemic inflammation might help reduce the severity of this severe skin inflammation. For antibiotic-associated colitis: Decreasing overall inflammation could help mitigate gut inflammation caused by antibiotic use. Antibiotics are often dispensed for short terms in response to particular infections. taVNS may be used prophylactically by some patients, such as with a once or twice daily regimen of 5-30 minutes of therapy, or more or less frequently when antibiotics are prescribed or treatment begins. Episodic use of taVNS may be implemented in response to symptom onset or identification instead, with usage of taVNS in a once or twice daily regimen of 5-30 minutes of therapy for a period of days to weeks, or until cessation of symptoms.

While primarily anti-inflammatory, long-term use of NSAIDs can paradoxically increase inflammation in some tissues. taVNS could potentially help with Gastritis and peptic ulcers: Reducing IL-6 and TNF-α levels may help decrease stomach and intestinal inflammation. For increased intestinal permeability: Lowering overall inflammation might help maintain gut barrier integrity. taVNS may be used prophylactically by some patients, such as with a once or twice daily regimen of 5-30 minutes of therapy, or more or less frequently. Episodic use of taVNS may be implemented in response to symptom onset or identification instead, with usage of taVNS in a once or twice daily regimen of 5-30 minutes of therapy for a period of days to weeks, or until cessation of symptoms. In other examples, taVNS may be provided when NSAID use is begun and/or changed, such as using taVNS for two weeks prophylactically when therapy begins.

Some medications used to treat Alzheimer's disease, particularly those targeting amyloid plaques, can have inflammatory side effects. taVNS could potentially help manage Amyloid-Related Imaging Abnormalities (ARIA) by reducing IL-6 and TNF-α levels may help decrease brain edema and microhemorrhages associated with these drugs. For brain swelling, lowering overall inflammation might help reduce cerebral edema. taVNS may be used prophylactically by some patients, such as with a once or twice daily regimen of 5-30 minutes of therapy, or more or less frequently. Episodic use of taVNS may be implemented in response to symptom onset or identification instead, with usage of taVNS in a once or twice daily regimen of 5-30 minutes of therapy for a period of days to weeks, or until cessation of symptoms. In other examples, taVNS may be provided when amyloid plaque therapy is begun and/or changed, such as using taVNS for two weeks prophylactically when therapy begins.

CAR-T Cell Therapy cancer treatments can cause significant inflammatory side effects. taVNS could potentially help manage Cytokine Release Syndrome (CRS) by reducing IL-6 and TNF-α levels may help mitigate the severity of CRS. For Immune Effector Cell-Associated Neurotoxicity Syndrome (ICANS), lowering overall inflammation might help reduce neurological side effects. For Macrophage Activation Syndrome (MAS)/Hemophagocytic Lymphohistiocytosis (HLH), decreasing inflammatory markers could help prevent or reduce the severity of these complications. taVNS may be used prophylactically by some patients, such as with a once or twice daily regimen of 5-30 minutes of therapy, or more or less frequently. Episodic use of taVNS may be implemented in response to symptom onset or identification instead, with usage of taVNS in a once or twice daily regimen of 5-30 minutes of therapy for a period of days to weeks, or until cessation of symptoms. In other examples, taVNS may be provided when CAR-T Cell Therapy is begun and/or changed, such as using taVNS for two weeks prophylactically.

For diabetes drugs, taVNS could potentially help with fluid retention and edema associated with thiazolidinediones (TZDs) by reducing overall inflammation might help mitigate these side effects. For infections associated with SGLT2 inhibitors, lowering systemic inflammation could potentially help reduce the risk or severity of genital mycotic infections and urinary tract infections. For pancreatitis associated with DPP-4 inhibitors and GLP-1 receptor agonists, reducing IL-6 and TNF-α levels may help decrease the risk or severity of pancreatic inflammation. taVNS may be used prophylactically by some diabetic patients receiving such therapies, such as with a once or twice daily regimen of 5-30 minutes of therapy, or more or less frequently. Episodic use of taVNS may be implemented in response to symptom onset or identification instead, with usage of taVNS in a once or twice daily regimen of 5-30 minutes of therapy for a period of days to weeks, or until cessation of symptoms. In other examples, taVNS may be provided when diabetic drug therapy is begun and/or changed, such as using taVNS for two weeks prophylactically.

In all these cases, the use of taVNS alongside drug therapies could potentially allow for more effective treatment with fewer side effects. By reducing key inflammatory markers like IL-6 and TNF-α, taVNS might help mitigate the inflammatory cascade that often underlies many drug-related side effects. This approach could be particularly beneficial for patients who are more susceptible to inflammatory side effects or those undergoing long-term treatment with drugs known to have inflammatory complications.

The auricular branch of the vagus nerve (ABVN) in the ear can be stimulated through various methods, primarily focused on non-invasive techniques. Transcutaneous electrical nerve stimulation (TENS) is a common approach, where electrodes are placed on the skin of the outer ear to deliver mild electrical pulses. Another method is transcutaneous vagus nerve stimulation (tVNS), which uses specialized ear clips or earbuds to target specific areas of the ear. Vibration therapy is also a potential option, utilizing mechanical stimulation to activate the ABVN. Manual stimulation techniques include acupuncture or acupressure applied to specific points on the ear. Some researchers have explored the use of low-level laser therapy (LLLT) to stimulate the ABVN. Additionally, there are emerging technologies such as ultrasound-based stimulation and magnetic stimulation that show promise for targeting the auricular branch. These methods vary in their invasiveness, effectiveness, and current level of scientific support, with electrical stimulation techniques being the most widely studied and applied, while vibration therapy is gaining attention as a potentially effective and non-invasive alternative.

As noted above, these drugs that can cause inflammatory side effects often have benefits that often outweigh the risks for many patients. Proper monitoring and management of side effects are crucial aspects of treatment with these medications. The ability to add a non-invasive and quick acting adjunct therapy using the auricular vagus nerve stimulation described herein would be of significant benefit to patients that experience these side effects. The auricular vagus nerve stimulation (AVNS) may be used as a response to observation of side effects, or may be used on a prophylactic basis. In some examples, a patient may be prescribed both AVNS and a drug or other therapy based on prior episodes of inflammatory response or perception on the part of the physician that the patient is susceptible to inflammatory response or may be particularly vulnerable to adverse outcome if an inflammatory side effect arises.

As used herein, a “medication” includes any of the drugs, antibiotics, T-cell therapy or any other substance that may be administered to a patient, transcutaneously, orally, intravenously or by any other approach, including from an implantable drug pump.

Transcutaneously delivered VNS may include electrical stimulation as described in several examples below. In other examples, mechanical (sonic, ultrasonic, or pressure based for example) stimulation may be applied. In still other examples, optical stimulation using light of selected frequency or having a band of frequencies may be delivered. While the following highlights the use of auricular-branch targeted VNS, other examples may target the vagus nerve in other locations, such as the neck of a patient, or at any location along the vagus nerve.

A VNS system may be used in the clinical or hospital setting, such as by use in an intensive care unit (ICU), as desired. The methods discussed herein may be performed using an implantable device in some examples. Other examples use a wearable device, such as the several examples of ear-worn devices which follow. Wearable devices may have a lower risk profile and do not require invasive surgery as with implantable systems. For example, some implantable systems require surgery to place a pulse generator, lead and cuff electrode, with the lead and cuff electrode positioned in the neck, a very sensitive location for the patient. The neck is also quite visible, so that any scarring resulting from surgery can be noticeable. Implantable devices have a finite lifetime, requiring subsequent surgery to replace or remove the pulse generator and other components. Implantable devices are also subject to migration or dislodgement, which can impair therapy and also present further risks, often requiring explant or further surgical intervention. A non-rechargeable implant will have a relatively shorter life, while rechargeable devices require recharging by the patient. Further, any surgery carries risks of complications, including infection in the acute setting as well as long term as infectious agents can be present in the surgical site and may not manifest for months or years after surgery. For all these reasons, while an implantable device may be used, a wearable system, including the ear-worn devices disclosed herein, may be preferable.

The stimulator could be operable via a smartphone or hand-held device that provides wireless connectivity to the stimulation device. While a stand-alone device (not connected to a hand-held) may be well suited for use in a hospital, a connected device (that is, one which can communicate by, for example, wireless connectivity to a smartphone, other handheld device, a server or computer via WiFi, etc.) may be better suited for use in a home environment. A software application on the hand-held device could be used to control stimulation (start/stop), monitor therapy, track therapy compliance of both the stimulator and companion drug therapy, and provide therapy compliance data to clinicians (as a data log or via communication over telecommunication or WiFi networks, such as the cloud).

Following are a number of illustrative wearable vagus nerve modulation devices which can be used for implementation of the methods disclosed herein. Further examples are also found in the following Patent Applications: PCT/US2025/032752, filed internationally on Jun. 6, 2025, titled WEARABLE DEVICES WITH ADJUSTMENT MECHANISMS, U.S. application Ser. No. 19/186,253, filed Apr. 22, 2025 and titled ADJUSTABLE EAR WORN APPARATUS, and U.S. application Ser. No. 19/355,071, filed Oct. 10, 2025, and titled ADJUSTABLE EAR WORN APPARATUS, the disclosures of which are incorporated herein by reference as showing further design details, including adjustable devices that will adjustably, mechanically, retain a position in the ear.

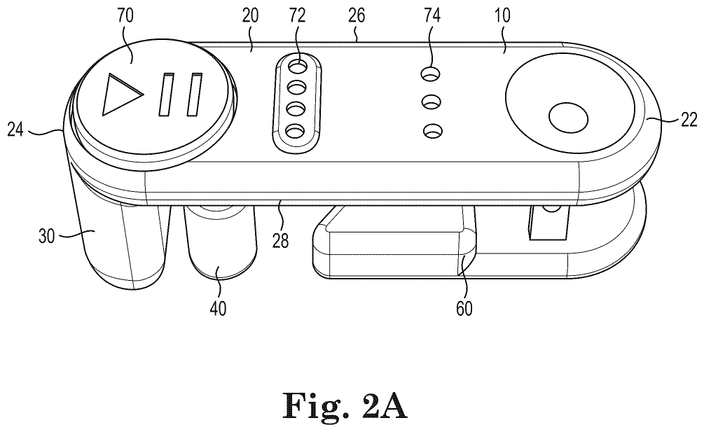

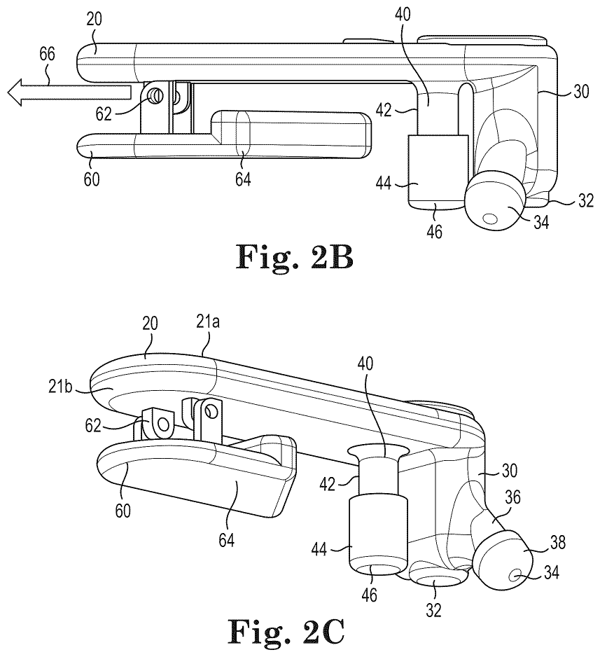

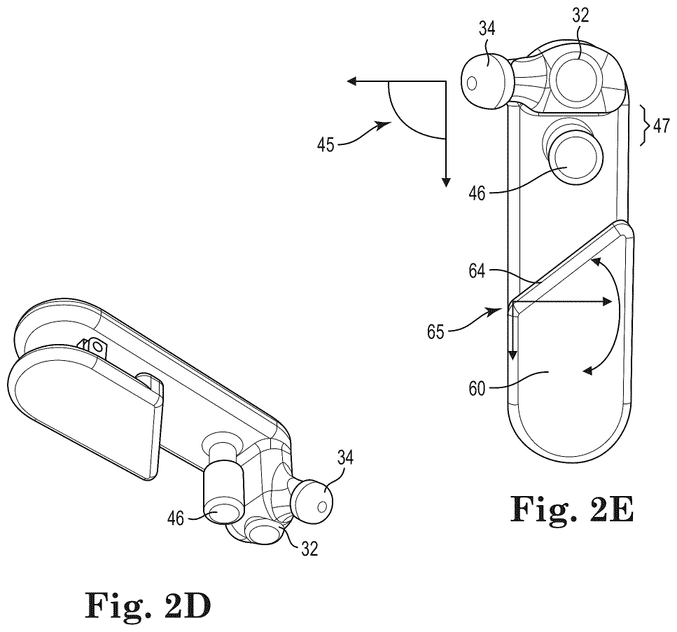

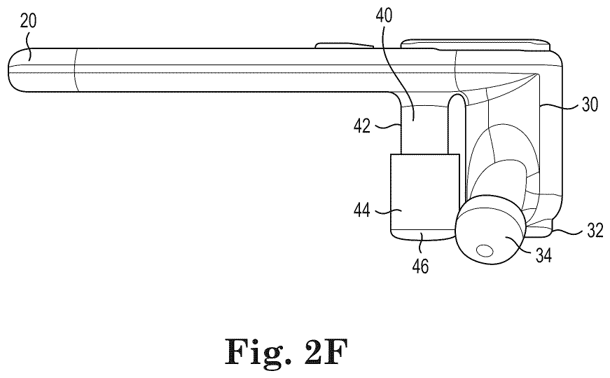

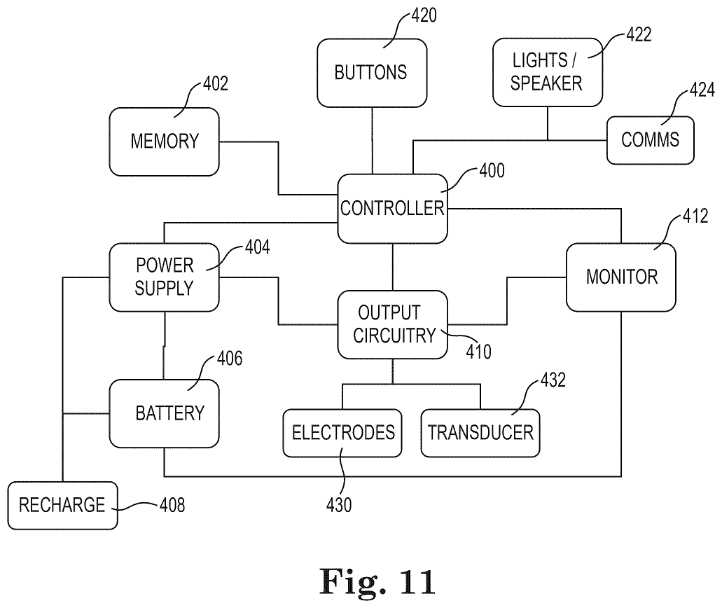

FIGS. 2A-2E are views of a first wearable vagus nerve modulation device. The device 10 is adapted for placement relative to an ear of a patient. A housing 20 contains electronics and a power source configured for providing output therapy energy, which may come in various forms including, in some examples, electrical pulses or other waveforms. Illustrative circuitry is shown and discussed relative to FIG. 11, below. The housing 20 has a length between a first end 22 and a second end 24, and a width between a first side or edge 26 and a second side or edge 28. The length is greater than the width in this example. For example, the length may be in the range of about 10 to about 40 millimeters, and width in the range of about 3 to about 15 millimeters, or more or less. The overall mass may be in the range of about 10 to about 50 grams, or more or less.

The device 10 includes a first extending structure 30 having a first end at the housing 20 and a second end apart from the housing. The first extending structure 30 has a length, generally in the range of about 3 to about 15 millimeters or more or less. At or near the second end of the first extending structure is an anchor arm 34 extending laterally therefrom. Optionally, the second end of the first extending structure may include a vaguest nerve stimulation element 32. In some examples, the device 10 may be characterized by the anchor arm 34 being configured to be positioned beneath the tragus when the device is placed. In some further examples, the anchor arm 34 is configured to be inserted into the external auditory canal of the patient, providing at least a first anchoring point for the device.

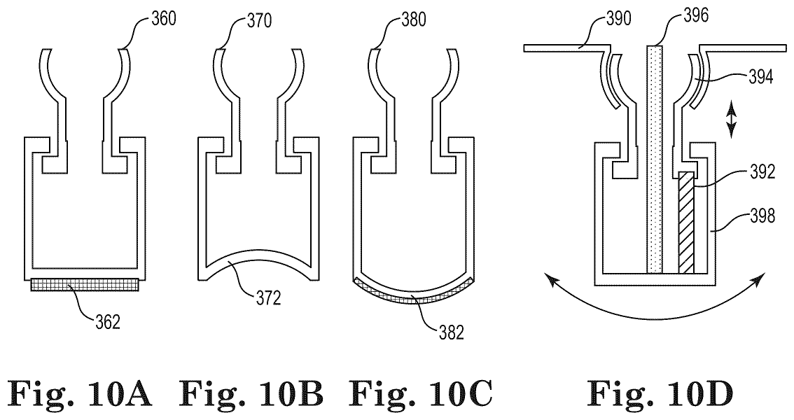

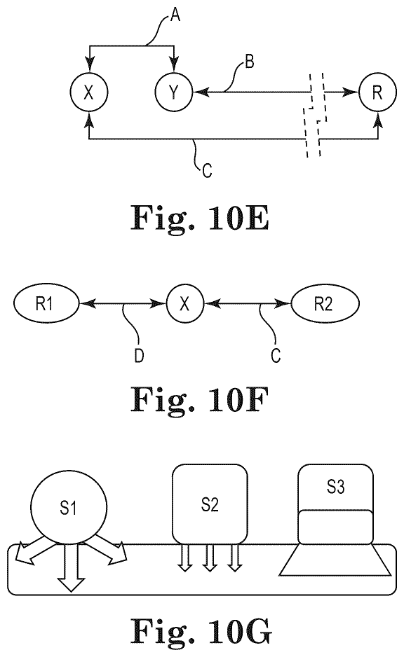

The housing 20 includes or is attached to a second extending structure 40 having a first end at the housing 20 and a second end apart from the housing 20. The second extending structure 40 has a length which may be in the range of about 3 to about 15 millimeters, or more or less. The length of the second extending structure may be variable, such as by having a spring structure or other biasing member or structure therein to allow flexibility when placed on a patient. The second extending structure 40 may have a variable shape, allowing for bending to a desired angle, and/or may rotate or pivot, so that the device can be adjusted to fit the user's ear. For example, FIG. 10D illustrates a variable length and pivoting mechanical structure; in other examples the structure itself may be flexible. As illustrated in FIGS. 2B-2D the second extending structure may have a first portion 42, fixed to the housing 20, and a second portion 44 sized and configured to move over the first portion 42.

In some examples, the housing 20 comprises molded pieces assembled together. For example, a first molded piece may be an upper lid and a second molded piece may be a lower container to which the upper lid attaches, thereby substantially forming the housing 20. Other manufacturing methods can be used. The first extending structure 30 may be a molded part of a lower container forming part of the housing 20, with the anchor arm 34 or a portion thereof included as part of the molding step, or attached thereto in a subsequent manufacturing step. The first portion 42 of the second extending structure 40 may also be part of the molded lower container of the housing 20, if desired. Other assembly or manufacturing methods can be used.

The example shown here includes a first electrode 32 on the first extending structure 30, and a second electrode 46 on the second extending structure 40. There may be more than one electrode in each of these locations, as shown in further examples below. Some examples may omit one, the other, or both of electrodes 32, 46. Rather than electrodes at 32, 46, devices for creating other therapy outputs (transducers, for example, for optical, mechanical/vibratory, magnetic, thermal or other therapies) may be used, in which case at least one transducer may be positioned on the first extending structure 30 and/or the second extending structure 40. Desirably, the positioning and/or degree of insertion of the anchor arm 34 may be such that the electrodes 32, 46 come into contact with the skin in the ear of the patient.

The anchor arm 34 may extend to a distance that is farther from the housing 20 than the electrode 32, as shown by FIG. 2B. As highlighted in FIG. 2C, the anchor arm 34 may include an expanded or bulbous end portion 38 coupled by a thinner portion 36 to the first extending structure 30. While a bulbous end portion 38 is shown, other shapes (oval, polygon, tapered, conical, etc.) may be used instead, and/or the end portion can be or include a foam material that can be compressed prior to placement, and then expands to secure the device in an anchored position. Alternatively, in other examples, the anchor arm 34 may have a consistent or tapered outer profile from its connection to the first extending structure 30 to its tip. The anchor arm 34 and end structure 38 may be a unitary or single piece, and may be hollow to allow audio signals to pass therethrough. In some examples, a speaker may be integrated into the device to deliver audio signals. The anchor arm 34 may also include one or more electrodes thereon. The anchor arm 34 and/or end structure 38 may be provided as a detachable/replaceable piece that can be selected from a range of sizes or shapes. In some examples, the anchor arm 34 and/or end structure 38 may be formed of a compliant material to conform to the space under the tragus and/or inside the auditory canal.

As shown in FIG. 2E, the anchor arm 34 may extend at an angle 45 relative direction of the length of the housing. In the example of FIGS. 2A-2E, the angle 45 is about ninety degrees, but in other examples it is envisioned that the angle 45 can be in the range of about 60 to about 120 degrees, or about 70 to about 110 degrees, or about 80 to about 100 degrees. In an example, the angle 45 of the anchor arm 34 may be adjustable, if desired, such as by use of a click-mechanism or flexible material to allow the arm to twist about the first extending structure 30. In still another example, the first extending structure 30 may be adjustable to twist about, for example, a central core, entirely or through a limited range of motion such as (using angle 45 as a guide) between about 60 to about 120 degrees, or more or less as desired. In the illustrative example shown in FIGS. 2A-2E, the position of the anchor arm 44 is fixed. One or more stops may be included to limit the translation, extension or rotation of the anchor arm 44, to the extent it is adjustable. The first and second extending structures 30, 40 may likewise be adjustable in terms of translation, extension/retraction and/or rotation, as desired.

The bulbous end 38 may be removeable or replaceable in some examples. In some examples, a plurality of sizes, shapes, and/or lengths for the bulbous end 38 may be available for use with the device 10. For example, the angle 45 shown in FIG. 2E may be adjustable by replacing the anchor arm 34 itself and selecting from a plurality of differently shaped, sized, or angled options. If desired, one or more through-openings or holes may be provided in anchor the arm 34 to allow air ingress/egress, facilitating hearing for the patient by avoiding complete blockage of the auditory canal. The anchor arm 34 and/or bulbous end 38 may further include one or more electrodes and/or transducers, either for therapy purposes or to enable or augment hearing of a patient. For example, a speaker may be provided, allowing the patient/user to hear audible indications of device and/or therapy status, to amplify sounds (as with a hearing aid), or to provide entertainment or communications to the patient/user.

In first example, when the device 10 is placed relative to the ear of the patient, the anchor arm 34 is positioned to extend beneath the tragus, while the electrode 46 is positioned at (i.e. in contact with) the conchae cymba, and the electrode 32 is positioned at (i.e. in contact with) the conchae caverna. In second example, when the device 10 is placed relative to the ear of the patient, the anchor arm 34 is positioned to extend beneath the tragus, while the electrodes 32, 46 are on opposing sides of the crus helix. In a third example, when the device 10 is placed relative to the ear of the patient, the anchor arm 34 is positioned to extend into the auditory canal, while the electrode 46 is positioned at (i.e. in contact with) the conchae cymba, and the electrode 32 is positioned at (i.e. in contact with) the conchae caverna. In an example, when the device 10 is placed relative to the ear of the patient, the anchor arm 34 is positioned to extend into the auditory canal, while the first and second electrodes 32, 46 are on opposing sides of the crus helix. These examples are not intended to be an exhaustive list of descriptions of the device positioning.

The electrodes 32, 46 may each have a surface area in the range of about 20 mm² to about 100 mm², or more or less. In some examples, each electrode as an area in the range of about 50 mm² to about 80 mm². The electrode 32 may be larger than the electrode 46 in some examples, allowing stimulation to be more targeted to the region of the electrode 46 by increasing the gradient of the voltage field in the vicinity of the electrode 46. The space or gap (edge to edge) 47 (FIG. 2E) between the electrodes 32, 46 may be in the range of about 2 mm to about 15 mm, or more or less. For example, electrodes 32, 46 may about 5 mm to about 10 mm apart (edge to edge). Voltage and/or current controlled output waveforms may be used, as further described below.

FIG. 2F shows an alternative approach. Here, the housing 20 still includes the first extending structure 30 and second extending structure 40, as with FIGS. 2A-2E. However, the clip structure is omitted here. Instead, the device can be secured in place by the extending structures themselves and the anchor arm 34. The second extending structure aids in holding the device without requiring user adjustment by having a variable length, using, for example, a spring loaded or otherwise adjustable connection between the upper part 42 and lower part 44 thereof, holding the electrode 46 on the skin of the patient while the anchor arm 34 holds the device in a desired position and secures the placement of the other electrode 32.

While the example of FIGS. 2A-2F is described as having electrodes as Vagus nerve stimulation elements, other devices, methods and/or modalities can be used. Examples may use any of optical stimulation with light sources (optical transducers) such as lasers (including vertical cavity emitting lasers) or light emitting diodes including, for example and without limitation, optical stimulation using wavelengths in the infrared, near-infrared, and/or visible spectrum. Other examples may use vibratory or acoustic stimulation with frequencies from relatively low levels (tens to hundreds of hertz) up to ultrasound frequency. Such stimulation may be described as mechanical stimulation, and may use a mechanical transducer to convert electrical energy to acoustic/vibratory energy using, for example a speaker or ultrasound generator. Some examples may use magnetic stimulation with electromagnetic fields generated using, for example, permanent magnets or electro-magnetic sources such as one or more inductive coils or other magnetic transducers.

Thermal stimulation may include heating of the nerve; heating may be achieved either by issuing higher frequency signals (RF heating), or by the use of a resistive heating element, for example and without limitation, wherein the resistive heating element may serve as a thermal transducer. Cooling may be provided, such as by having a removeable/replaceable thermal element that can be placed in a refrigerator prior to use, by including a Peltier cooling apparatus, or by having channels allowing cooling fluid to be circulated, either of which may be a thermal transducer. Thus, rather than the electrodes described above, one or more transducers can be used to convert stored power (usually electrical power from a battery) to a different energy modality. Each of these methods offers unique advantages and may be tailored to specific applications based on factors such as precision, invasiveness, and compatibility with the nerve tissue. For example, optical stimulation offers precise control over the timing and location of nerve activation. Acoustic and magnetic stimulation techniques can penetrate deeper tissues and may be non-invasive, making them suitable for certain clinical scenarios. Thermal stimulation, on the other hand, can modulate nerve activity by altering temperature gradients within the tissue.

In some examples, a combination of modalities can be used. For example, thermal stimulation may be generated by the use of higher frequency (RF) outputs from electrodes, paired with lower frequency pulsed electrical field outputs at frequencies in the tens to hundreds of hertz. Such signal combinations may be delivered in an overlapping or simultaneous manner, or the device may cycle between one therapy mode and another, as desired. Electrical stimulation can also be paired with magnetic, acoustic/vibratory, and/or optical stimulation. Other combinations can be used as well.

Separate therapy may also be provided, such as with the delivery of anti-inflammatory or other medications to the patient along with the issuance of stimulation signals, or by also providing circulatory or respiratory support to the patient and/or additional stimulation signals, or thermal controls such as inducing therapeutic hypothermia or other temperature management. In some examples, therapy combined with an analgesic to ensure that the patient will not feel the therapy delivered by the stimulation device. An analgesic may be systemically delivered (injection, oral, etc.) or may be locally delivered such as by elution from the electrode surfaces or by using a gel or liquid containing analgesic substances (such as lidocaine) on the electrode surfaces.

The device may coordinate therapy delivery with other actions. In some examples, the device may be commanded to start a therapy session, while another therapeutic activity is ongoing, such as having the patient engage in a memory game or other activity while therapy is being delivered. Coordinated timing can be facilitated by use of the controls on the device itself, or the device may include communications circuitry (such as a Bluetooth or Bluetooth Low Energy antenna and chip) to communicate with a programming device or smartphone having counterpart communications circuitry; an application operating on the programming device or smartphone can be used to start therapy at a desired time. Other coordination may include the use of biological signals. Heart rate, for example, can be monitored by the device itself (such as by adding or including an earlobe clip), or by a second device such as a cardiac monitor; when the heart rate is above a threshold, such as a threshold in the range of 100 to 140 beats per minute (or other setting), the patient may be experiencing a high degree of inflammatory response, so therapy can be turned on in response to elevated heart rate. On the other hand, if the heart rate becomes bradycardic, such as below about 40 to 60 beats per minute, therapy may be stopped. In another example, pupillometry can be used to turn therapy on or off by obtaining an image of the eye, using a smartphone or other device having a camera, and modulating or turning therapy on or off in response to the results of such measurements. Synchronization to other therapies, including physical therapy, drug delivery, or any other intervention can be useful to augment the patient's response to other therapies by Vagus nerve stimulation.

The nerve stimulation elements of the device can be designed to be modular, allowing for easy customization and adaptability to individual patient needs. This feature enables healthcare professionals to easily change out the stimulation elements as necessary, providing a tailored and optimized treatment plan for each patient. The modular design enhances the versatility and flexibility of the device, ensuring that it can be easily adjusted to accommodate different therapeutic and anatomic requirements. This approach enhances the clinical utility of the device, offering a personalized and effective treatment option for patients with varying neurological conditions. Modularity may be provided by, for example, providing aspects of the device housing and/or neural stimulation elements in the system in a range of sizes or types. For example, if electrodes are used to issue electrical stimuli, the electrodes may come in different sizes (surface areas) and/or shapes, which may be selected and/or replaced. Aspects of the housing and the extending structures can also be adjustable or replaceable to accommodate different anatomies (larger or smaller ears), including, for example, pediatric sized systems for smaller ears. The system itself may come in a range of sizes, if desired.

The device may be programmable or reprogrammable, such as by plug-in-type attachment to a port located on the device, or by use of magnetic/inductive, wireless (RF, such as Bluetooth) communication, optical communication, or by having one or more buttons, dials, or other user-accessible controls accessible on the device. To this end, as discussed further below with reference to FIG. 11, a communications circuitry may be included in the device.

Optionally, the example in FIGS. 2A-2E also includes a clip 60 for clipping the device 10 into a desired position in the ear of a patient. The clip is secured with a spring-loaded hinge 62 that allows the clip body 64 to be pivoted away from the housing 20, allowing the helix of the patient's ear to be captured between the clip body 64 and the housing 20, securing the device 10 into the ear. Any resilient or force generating structure can be used in place of a spring-loaded hinge, such as a magnet, a hinge made of flexible material, etc. In a further example, the hinge 62 may be coupled to the housing 20 using a sliding or ratcheting extension to accommodate different sized ears of different patients, by allowing hinge 62 to move in direction 66 (FIG. 2B) along one or more interlocking tabs extending from the housing 20. The clip 60 may be configured for positioning over any portion of the outer ear, that is, at the top, side, or bottom, as desired. Clipping onto the top may provide the greatest amount of ear tissue for such use, while clipping to the side can provide mechanical stability opposing forward rotation of the device as the anchor arm opposes opposite rotation.

The materials used throughout may include any material suitable for skin contact for an extended period of time (hours, days or even weeks). For an electrical stimulation system, the electrodes 32, 46, for example, may be made of any of graphene, titanium, nickel titanium (nitinol), platinum, platinum-iridium, gold, silver, stainless steel (including MP35N alloy) or any other metal or conductive polymer or other material that can be worn on the skin. Coating layer(s) may be provided to optimize tissue interface characteristics, as desired. As illustrated in FIGS. 10A-10D, below, the electrodes may be configured to receive or carry thereon a conductive material, such as a gel, hydrogel, or other tissue interface component. Pads may be attached if desired. Alternatively, dry electrodes can be used, if desired. The other tissue contacting portions of the device 10 may be made of suitable plastics, silicone, etc. adapted for wear on the skin of a patient/user.

Biocompatible materials may be selected to enhance conduction of the therapy signal between the electrode or therapy generating element and the tissue (electrical conduction, mechanical conduction, optical transmission, etc). Biocompatible materials may also be selected to enhance adhesion of the therapy generating element and the tissue. Biocompatible materials may also be selected to provide an analgesic effect to suppress perception of the therapy. All types of materials may also be combined into a single material. Materials may be attachably and detachably connected to the therapy-generating element. For example, hydrogel pads may be replaced. In cases of wet materials, one or more moisture barriers (e.g. metal foil) may be used for packaging and temporarily adhered over the material to preserve functionality for extended shelf life. In other examples, materials may be separately packaged within a preserving pouch, packet, or container and applied prior to use. In some cases, the material may include a barrier material or membrane that is removed prior to use. In some cases the barrier material or membrane may include extensions, tabs, buttons, or other structures to aid in handling the material while attaching the material or removing the material from the device.

Returning to FIG. 2A, the housing 20 optionally includes progress indicator lights at 72, and alert lights at 74. If desired, a speaker may be included in or on the housing 20 as well and used for issuing audible alerts, instructions for use, device status, or other purposes such as for providing an audible signal for entertainment or relaxation purposes (playing music for example). The progress indicator lights 72 may be light emitting diodes (LEDs) or any other suitable light generator, as desired. Upon powering the device, it may take some period of time (seconds to minutes) for electronic circuits to convert voltage levels from the power source (e.g. battery) to levels suitable to power the therapy driving sub-circuits. For example, a boost converter, or inverter may require time to charge up capacitors, or circuit elements that can supply voltage and or current draw more readily than a battery (e.g. coin cell, which has a limited voltage and current draw). An indicator may be used to indicate when the device is ready for use, such as when a capacitor used for storing power to be used in therapy is charged to a desired level. As the device is preparing itself, a different indicator may be used, such as having a flashing or blinking light during preparation of the device, which turns solid once the device is ready.

When therapy is being delivered, the progress indicator lights 72 may light up one at a time to indicate progress of a therapy regimen, which may, for example, have a duration in the range of about 1 to about 60 minutes, or about 5 to about 30 minutes, or about 20 minutes. A liquid crystal display, touchscreen, or other display may be used instead, if desired. Alert lights 74 may include, for example, lights that indicate problems with the device, which may include poor contact with the skin (determined for example using a temperature sensor or an impedance monitor, as desired), expiration of the device, other failure in the device, low battery, etc. A button at 70 may be used to initiate or pause the device therapy, as desired.

Electrical output therapy parameters may include, for example, issuance of square wave pulses that are either current-controlled or voltage controlled, as desired, issued at a pulse repetition rate in the range of about 1 to about 200 Hz, or about 10 to about 50 Hz, or about 20 Hz, 30 Hz, or 40 Hz, or more or less. Frequency may be adjustable or selectable, or it may be fixed. Current controlled pulses may be, for example, issued at an amplitude in the range of about 0.1 mA to about 20 mA, or about 0.4 mA to about 12 mA, or about 8 mA. Voltage controlled pulses may be, for example, in the range of about 1 mV to about 15 V, for example and without limitation. Peak voltages, either stored within the device or used for therapy outputs, may be in the range of up to about 50 V or higher.

Current or voltage may be adjustable, if desired, such as by providing wireless control in which the device 10 contains a transceiver, such as a Bluetooth chip and antenna, to be programmed by an external device such as a smartphone or tablet operating an application dedicated to the vagus nerve system. Other communication means can be used, including optical, magnetic/inductive, vibratory, etc., as desired. Alternatively, one or more buttons on the device may be used to increase or decrease output amplitude, as desired; additional indicators on the device may be used to allow amplitude settings to be determined visually. Some systems may, on the other hand, be pre-programmed with limited or no therapy adjustments available.

Electrical pulses may be delivered using, for example a pulse width in the range of about 10 microseconds to about 20 milliseconds, or more or less; pulse width may be adjustable or selectable, as desired. Some examples may use pulse width in the range of about 100 microseconds to about 500 microseconds. An illustrative example uses a pulse width of 250 microseconds, repetition rate of 20 Hz, and amplitude of 8 mA, delivered in a 20-minute session, once a day or twice a day, for up to two weeks, for example. More frequent sessions, or less frequent sessions, and shorter or longer sessions, and/or longer or shorter regimens (one day to several weeks, for example) may be used. For example, twice-daily, once-daily, or alternating day therapy can be used for period of weeks or months, or longer, depending on patient needs.

Other signals may be used, including burst outputs (having closely grouped pulses in time separated by longer quiescent periods), or shaped outputs (triangular, ramped, descending, etc.) and/or sinusoidal signals. Some examples deliver stimulus using a waveform without any underlying carrier wave. Others may use, for example, a carrier wave in with a frequency in the kHz to MHz range. Interferential stimulation may be used as well, in which two therapy signals (such as sinusoidal outputs) are delivered at different frequencies, resulting in the effective delivery of a beat frequency (the difference between the therapy signal frequencies) arriving at target tissue.

In some examples, a subperception therapy is delivered. For example, with an adjustable electrical stimulus system, any of amplitude, pulse width, or repetition rate may be adjusted until paresthesia (tingling) or other sensations are observed/reported by the patient, and then the controlled parameter (amplitude, pulse width, or repetition rate) is reduced. In one example, amplitude is increased until paresthesia is observed, indicating that the sensory threshold has been reached, and then therapy amplitude is set by reducing the amplitude by a fixed amount or a percentage relative to the sensory threshold. Pulse width may be increased until paresthesia is observed, indicating that the sensory threshold has been reached, and then pulse width is set by reducing by a fixed quantity or percentage. Other combinations may be used to achieve a sub-perception therapy. Supra-perception therapy may be used if desired, as a way of providing the patient feedback that the therapy is on and in use.

In some examples, therapy may be set by use of a population-based control and/or controls adjusted using patient characteristics. Closed loop sensing may not be available for some systems. A therapy setting for any of electrical, optical, mechanical (acoustic or vibration), and/or magnetic stimulation can be selected using amplitude, frequency/wavelength and/or other characteristics after testing in a population of test subjects. The therapy setting may be chosen provide effective therapy with minimized side effects based on the results of such testing.

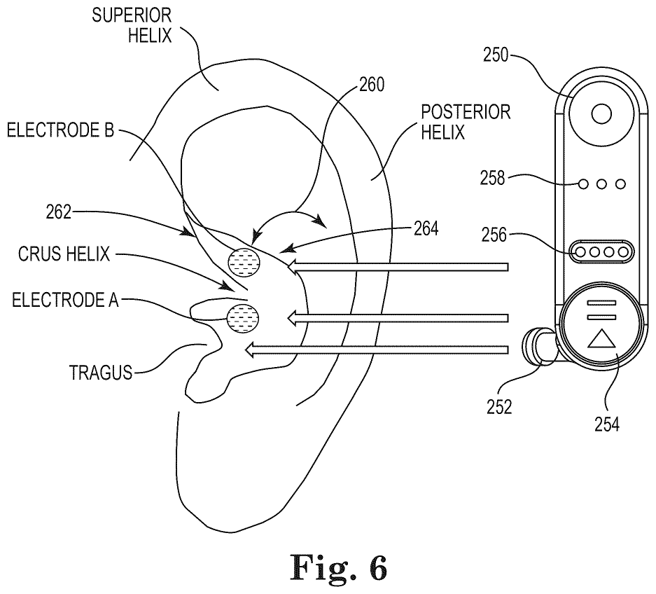

Some of the preceding examples indicate the use or possibility of reshapeable first extending structure 30 and/or second extending structure 40, or an anchor arm 36 which is reshapeable. Other examples make each of these pieces a rigid element not allowing for reshaping. In some examples, a rigid second extending structure 40 has a spring or other resilient member therein allowing the length to vary in response to patient anatomy. The device may then be placed by inserting the anchor arm 36 with its end 34 in the auditory canal of the patient, and then twisting the device to bring the second extending structure into contact with the conchae cymba. The twisting movement may be as indicated by arrow and line 260 in FIG. 6, below, until the second extending structure 40 abuts the crus helix or antihelix, as explained relative to FIG. 6.

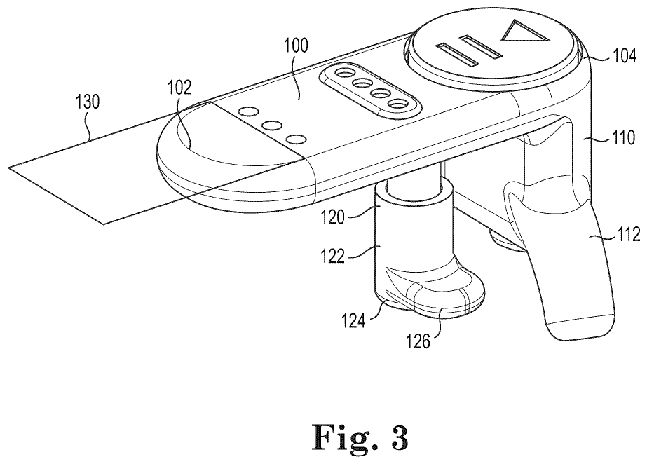

FIG. 3 is a view of a second wearable vagus nerve modulation device. This device has several features distinct from that of FIGS. 2A-2D. The device includes a housing 100 having a first end 102 and a second end 104, defining a length therebetween in the range of about 15 to about 30 millimeters, or more or less. At the first end 102 of the housing, a tab of adhesive tape 130 extends. The tape 130 can be used to secure the device in place in the ear of a patient by being extended over and around the top, side, or lower portion of the ear. Prior to placement, the tape 130 may have a removable tab covering and preserving the adhesive portion thereof. The tape 130 may extend for any suitable length, such as in the range of about 10 millimeters to about 50 millimeters, or about 20 to about 40 millimeters, or about 30 millimeters, as desired. Any suitable medical grade tape can be used at 130. Rather than a tape, a flexible substrate of any suitable material may be used, with adhesive thereon. The tape or flexible substrate may be removed from the device if desired and/or replaced from time to time.

Further on the under-side of housing 100, a first extending structure 110 is shown. The first extending structure 110 has an anchor arm 112. In this instance, the anchor arm 112 is at an angle of about 75 degrees relative to the edge of the housing 100. The outer profile of the anchor arm 112 can be seen to be generally cylindrical, and may include a taper, if desired, in contract with the expanded end shown in the example of FIG. 2A-2E. A stimulation element may be positioned along the bottom of the first extending structure 110, though this is not marked in the figure.

The housing 100 also includes a second extending structure 120. The length of the second extending structure 120 may be variable by using a spring-loaded design to move the lower portion thereof, shown at 122. Another stimulation element is shown at 124, and may be an electrode or a transducer for any of optical, mechanical (acoustic or vibration), and/or magnetic stimulation/modulation. The two stimulating elements may be of the same type, or may each provide a different modality of therapy, such as by pairing an electrode on the first extending structure 110 with a mechanical or optical transducer on the second extending structure 120, or any other desired combination.

A wing or extension 126 is shown on the side of the lower portion 122, and may be used to interact with the patient anatomy to help hold the device 100 in position, for example, with the wing 126 fitting under the helix and/or inferior crus of the patient or, in the alternative, under the cymba; to this end, the lower portion 122 of the second extending structure 120 may be adjustable, such as by rotation/partial rotation, to allow the wing to be directed as desired. Alternatively, the wing 126 may be rotatable relative to the lower portion 122 and/or the second extending structure 120.

Either or both of the extending structures can be formed of deformable or flexible materials, if desired. This may permit changes in extension, rotation or translation of the extending structures relative to the patient's anatomy. A limiting structure may be provided, such as grooves, stops, rails, etc. to limit any of extension, rotation and/or translation.

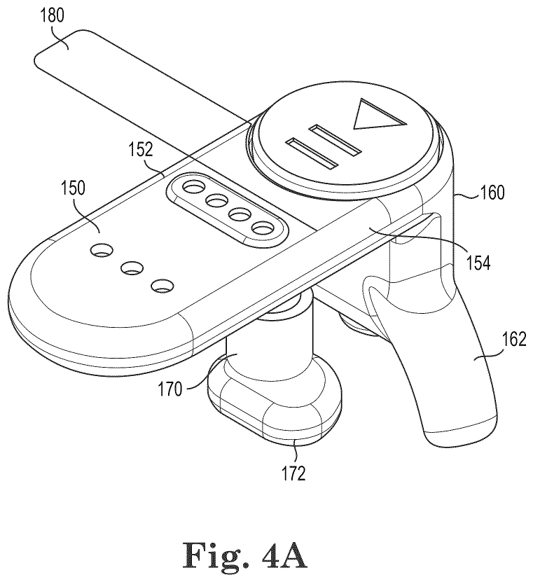

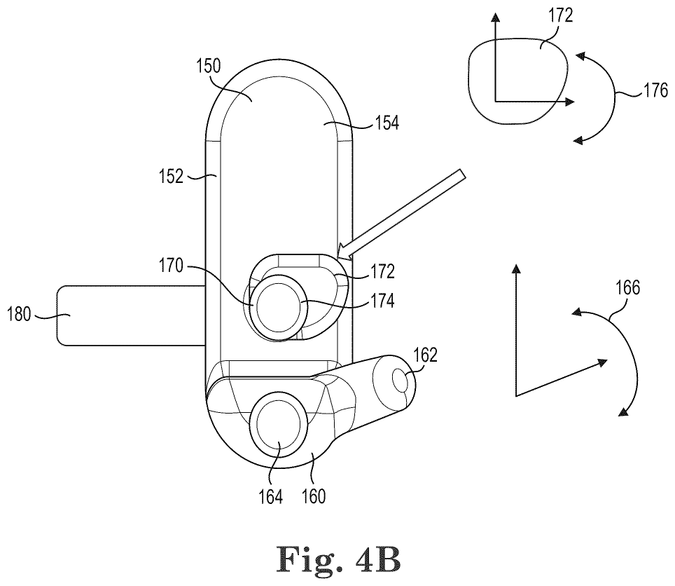

FIGS. 4A-4B are views of a third wearable vagus nerve modulation device. Here, the housing 150 includes a first edge 152 and a second edge 154, defining the width of the housing 150 therebetween. A tab of adhesive tape 180 extends from the first edge 152, and can be used to secure the device into a desired position on the patient's ear. The tape 180 extends opposite of the edge 154 from which the anchor arm 162 extends from the first extending structure 160, providing opposition to the anchor arm 162 to secure the stimulating elements down onto target tissue.

The second extending structure 170 can be seen to again include a wing or tab 172. While the wing 126 of FIG. 3 extended in one direction, it can be seen that the wing 172 now extends around the second extending structure 170 in multiple directions. The view from below in FIG. 4B shows how the wing 172 extends in multiple directions away from the second extending structure 170, while the second stimulating element 174 is positioned separately from the wing. Again, the lower portion of the second extending structure 170 may be rotatable if desired and/or the wing 172 may be rotatable.

The anchor arm 162 can also be observed as extending at an acute angle relative to the edge 154, as also indicated in FIG. 4B. The anchor arm 162, or a portion thereof, may be attachable or detachable from the extending structure, may be available in a range of sizes or shapes, and/or may be made of compliant material to fit varying ear shapes. The extending structure and/or anchor arm may also include therapy delivering elements. In some examples, the tape 180 may instead be a flexible substrate that can be formed into a shape desirable for holding the device in place. For example, a wire, with or without shape memory, optionally including a soft polymer or other cover, may be used to create tension against an anatomical structure of the ear for holding the device in a desired position.

FIG. 4B further illustrates the inclusion of a first stimulating element at 164, and the second stimulating element at 174, each of which can be an electrode or other transducer as described elsewhere herein. In some examples, the anchor arm 162 may be adjustable within a range of available angles as illustrated at 166, for example, in a range of about 45 degrees to about 135 degrees for the angle illustrated at 166, relative to an axial length of the overall device 150.

In some examples, the wing 172 may be rotatable, at least partially, about an angle 176 as shown in the inset. The range of rotation may be anywhere from about 10 degrees to about 90 degrees from the position shown. Thus the wing 172 may rotate toward the first extending structure 160, without being allowed to contact the first extending structure. For example, the size of the wing 172 may be such that it can rotate without contacting the first extending structure, or the range of movement of the wing 172 may be limited. The wing 172 may, for example, be positioned beneath the helix and/or inferior crus of the patient or, in the alternative, under the cymba. In some examples the wing 172 is non-conductive and used only to support positioning of the device. In other examples, the wing 172 may be a conductive element, serving as an additional electrode usable in combination with other electrodes in the system, if desired.

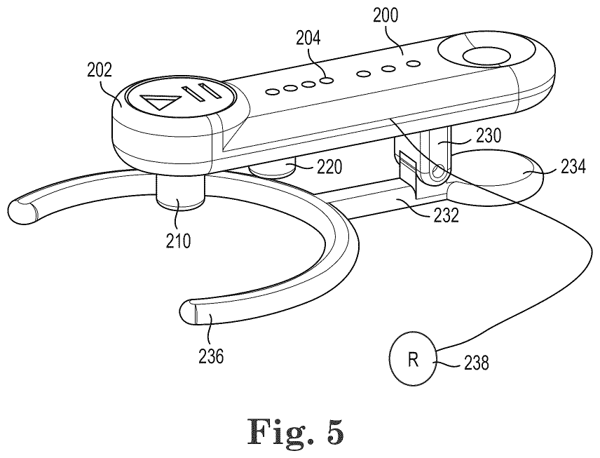

FIG. 5 is a view of a fourth wearable vagus nerve modulation device. In this example, the housing 200 has a first extending structure 210 and a second extending structure 220, shown in different footprints than before. One end of the housing 202 is expanded to accommodate batteries and electronics, rather than having those elements disposed partly in the first extending structure, as may optionally be the case in the versions shown in FIGS. 2A-2E, 3 and 4A-4B. An anchor arm (not shown) may be included for aiding in the positioning and retention of the device or, in the alternative, the anchor arm may be omitted in this example. The indicator and/or alert lights 204 are shown in row extending longitudinally, rather than across, the housing 200. These modifications can appear in any of the previously shown versions of the device using other mechanisms and/or tape for holding the device in place in the ear of a patient.

This version of the device 200 includes a positioning loop 236, attached to a bar 232 that couples to a hinge 230. The loop 236 is designed to wrap around the base of the ear where the ear attaches to the scalp, in order to hold the device 200 in a desired position on the patient. That is, the loop 236 is positioned to partly wrap about the ear adjacent the skull. The tab at 234 can be used to manipulate the hinge 230 to pivot the housing 200 back while placing the loop 236. In some examples, the loop may not be attached by a hinge, and may be formed of a compliant material. The loop 236 may be removeable and replaceable with varying sizes and/or shapes. The loop 236 may include adhesive. The loop 236 and/or the clip may incorporate one or more stimulation elements, such as an electrode (or plural electrodes) positioned on the loop.

A remote return electrode 238 can be provided if desired, as shown in the example of FIG. 5. A return electrode 238 can be added to any of the examples in FIGS. 2A-2E, 3 and 4A-4B, as well the systems that follow. The return electrode 238 can be used as described below with reference to FIGS. 10E-10F, and is optional. For example, the return electrode 238 may include an adhesive coating or may be held in place using tape at a desired position on the head, neck, torso or elsewhere on the patient's body, and is coupled by wire to the housing 200.

FIG. 6 illustrates placement of a wearable vagus nerve modulation device. The device 250 includes an anchor arm 252. A button 254 is provided for use in activating, pausing or stopping therapy. A tap design, rather than button 254, may be used if desired. Indicator LEDs may be provided at 256 and/or 258. It may be noted that while the indicator lights 256/258 may be useful in the context of a patient having a caregiver, these may be of less utility for a patient using a device 250 at home. A speaker may be used to provide audible indications of status, such as by inclusion on the anchor arm 252 or elsewhere on the device.

The system of FIG. 6 is illustrated and described as providing electrical stimulation, though other modalities of therapy can be used instead. The device 250, with anchor arm 252, and first and second electrodes as in any of the preceding versions of a wearable vagus nerve modulation device, will be placed as indicated by the arrows. The anchor arm 252 passes behind the tragus, and/or into the auditory canal. This brings the first electrode to the position marked Electrode A, and the second electrode to the position marked Electrode B. Such positioning would also put the first electrode on/at the conchae caverna, and the second electrode on/at the conchae cymba. In other examples, one or the other of the electrodes may be differently placed, and/or more than two electrodes can be used. Further, rather than electrodes, other vagus nerve stimulation elements may be used, as desired, singly or in combinations.

As indicated by line/arrow 260, in several examples the device may be positioned by inserting the anchor arm 252 into the auditory canal, and/or beneath the tragus, and then twisting the device. Some examples may twist the device in a superior/anterior direction, bringing the second extending structure (not shown, but carrying Electrode B in several examples) into a position abutting the anatomy of the exterior of the ear, such as a superior portion at the posterior edge of the crus helix, marked at 262. This positions the housing more vertically in the ear, with the end opposite the anchor arm 252 near the superior helix. Other examples twist in the opposite direction, in an inferior/posterior direction, bringing the second extending structure (again, not shown, but carrying Electrode B in several examples) into a position abutting the anatomy of the exterior of the ear, such as the antihelix, as indicated at 264.

This twisting step highlighted at 260 works the device into a desired position, and can be performed by the patient or a caregiver, such as an ICU nurse, in a simple, quick installation step. In some examples, no molding, curing or reshaping is needed. Because the second extending structure has a variable length, such as by including therein a resilient member or spring, such twisting allows the device to more or less automatically achieve a desirable position in which the electrodes or other actuator(s) are positioned against the patient's skin. A wing, such as shown at 172 in FIG. 4B, or at 126 in FIG. 3, helps hold the device in the desired position as well. An adhesive strip, such as tape or other substrate material, can be added if needed to maintain device positioning, however it is envisioned that an additional piece of tap will not be needed for most patients, again simplifying the use of the system for the patient and medical personnel.

If a clip is used as in FIGS. 2A-2E, the clip would pass over the helix, for example at a superior or posterior location, or elsewhere and/or in-between, to hold the device in place. If tape is used as in FIG. 3, the tape may extend to and over the region marked superior helix to hold the device in position. If tape is used as in FIGS. 4A-4B, the tape would extend to and over the region marked posterior helix.

Regardless the clip or tape inclusion, the device 250, using the anchor arm 252 and the extending structures described herein, is configured for placement such that the entire device, in some examples, is positioned inside the periphery of the ear, with no wires extending therefrom. In other examples, a wire does extend out to a return electrode positioned elsewhere on the patient, such as the torso or neck, if desired. In some examples, only a single device 250 is present in the system, omitting a second device positioned on the other ear. The device 250 may be configured for positioning on the left ear, as may be inferred from FIG. 6. Alternatively, the device may be configured for positioning on the right ear, if desired.

Some examples may include two devices 250 that are separately positioned, without mechanical/electrical contact therebetween, one for each ear of a patient. For such as “two-device” system, therapy can be delivered independently by each device, in some examples. In other examples, may be coordinated such as by providing wireless communication circuitry in each device so that the two devices can communicate with one another to coordinate therapy delivery, or so that each device can communicate with a programming device, such as a dedicated programmer or a user's smartphone (operating an application specific to the system) that communicates with each device to synchronize therapy delivery. “Synchronized” therapy may include any of delivering therapy with pulses delivered at the same time, or with pulses interleaving, or with pulse trains overlapping, or with pulse trains from each device alternating with one another so that only one device is actively issuing output at any given time, or actively issuing an output pulse train at any one time.

Other examples may use a very lightweight version of the device 250, omitting electronics therein, and coupling to a separately positioned pulse generator such as by wired connection thereto, where the output of the pulse generator provides power and defines the pulsed outputs of the device.

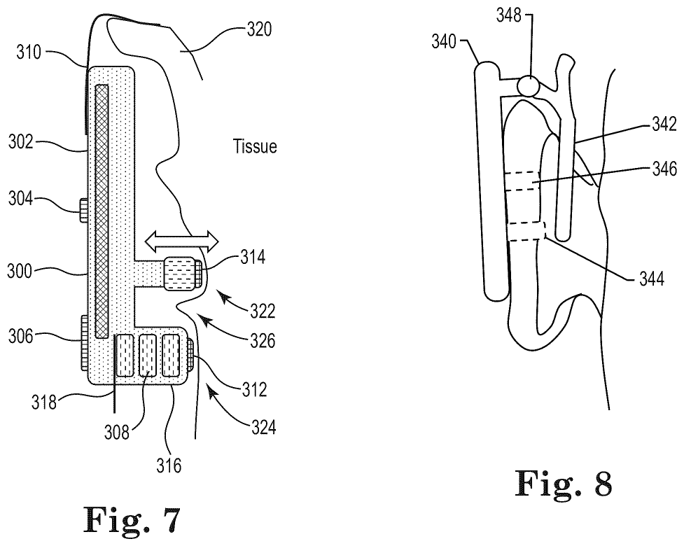

FIGS. 7-8 are side section views illustrating electrode contact and mechanisms for securing a wearable vagus nerve modulation device to the ear. In FIG. 7, a device 300 is shown secured to the patient's ear using tape 310 attached to the device 300 and the superior helix 320. The device is sized and shaped so that when positioned as shown, the first electrode 312 is positioned at the conchae caverna 324, and the second electrode 314 rests against tissue at the conchae cymba 322. The positioning can also be characterized as having the first electrode 312 and second electrode 314 positioned on opposing sides of the crus of helix 326 The anchor arm is not shown as it would be out of the plane of the view shown, but is understood as being positioned beneath the tragus and/or in the auditory canal when the device is placed as shown in FIG. 7.

Illustratively, and without limitation to a particular layout, the device 300 is shown having a printed circuit board 302 therein, coupled by feedthrough or other wires (not shown) to the alert indicators 304, on/off/pause button 306, first electrode 312 and second electrode 314. A stack of battery cells 308, which may be standard button cells or may be a custom design, is contained in this example in the first extending structure 316. Other layouts and battery types can be used; any number of battery cells may be used, though it is expected generally that one to three cells would be used. The device may be a single use device (where single use means use for a single patient for a limited period of time, such as a single therapy session, or repeated therapy sessions for up to one month, or up to fifteen days, for example, and/or where single use indicates the batteries 308 are not replaceable). In other examples, the device may have rechargeable or replaceable batteries 308 and is adapted for chronic use. A removeable tab 318 may be used to preserve battery capacity prior to use; once the tab 318 is removed, the electrical circuit for powering the device is completed and the device electronics are enabled. In some examples, an optical light pipe such as an optical fiber may be used to transmit light from LEDs on the circuit board 302 to desired positions, either for use on the indicators, or if used as therapy delivery devices, the optical fiber may extend to at least one of the stimulation elements 312/314, which would in turn be optical elements allowing light energy to pass therethrough, for example, lenses. If a light pipe is used, the “stimulation element” may be understood as including each of the LED or other optical source on the circuit board, the light pipe or fiber, and any interface element such as a lens that delivers light from the light pipe or fiber to tissue at a target location.

FIG. 8 illustrates another example. Here, the device 340 includes a clip 342 that wraps around the ear, anchoring the device in position. As a result, the first stimulation element 344 and second stimulation element 346 are held against tissue at the desired positions. The stimulation elements 344, 346 may be electrodes or may be transducers as described herein. A hinge 348, which may be spring loaded, is used to secure the device in desired position. An anchor arm as described and shown in previous examples may be used to provide an anchor against the tragus and/or inside the auditory canal.

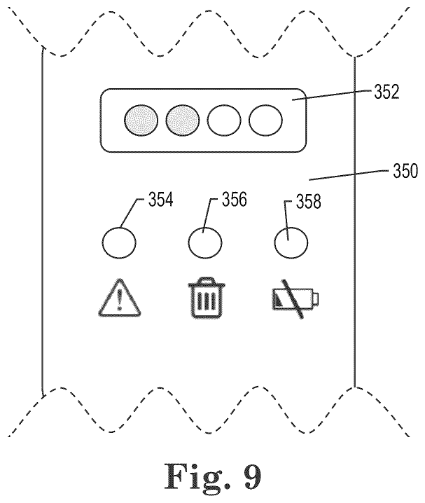

FIG. 9 provides an illustrative example of status and warning lights. The illustrative device 350 includes progress or status lights 352; as shown, therapy is in the range of about halfway to completion. Alert indicators (lights typically) can include a contact alert 354, an end of life or catastrophic failure alert 356, and a low battery alert 358. Other alerts and mechanisms for interaction with the user may be used. A digital screen can be used if desired instead of discrete alert lights.

FIGS. 10A-10G show illustrative stimulation device designs. FIG. 10A shows an electrode assembly at 360, having a flat contact surface on which a hydrogel or other tissue coupling element 362 is provided. An adhesive, for example, may attach the coupling element 362 to the electrode assembly 360. FIG. 10B shows an alternative 370 with a concave contact region 372, which may help hold/contain the tissue coupling element and/or a hydrogel or other gel for aiding in signal transmission across the tissue-stimulation element interface. In some instances, an electrode may have a surface that is textured, roughened, scored, or dimpled to increase effective surface area, to thereby lower impedance, in addition serving to receive or retain coupling material, gel, and/or adhesive. Not only electrodes, but any of the described vagus nerve stimulating elements may have textured, roughened, scored or dimpled surfaces, or may comprise an adhesive layer, or may receive a piece of coupling material, gel or adhesive thereon. FIG. 10C shows an alternative 380 having a convex contact surface 382.

FIG. 10D highlights two different ways that the electrode 398 contact can be made adjustable. A spring or other resilient member 392 or 396 can be used to maintain pressure against the tissue once the device 390 is placed. The resilient member 392 or 396 may be, for example, a coiled spring, a compressible foam, or any other suitable structure able to be compressed and expand after removal of applied force. A swivel or ball-joint structure shown at 394 can allow the electrode to be laterally angled as desired. The resilient member shown at 392 presses against the end of the swivel or ball-joint structure 394; alternatively, the resilient member 396 may be used instead and is shown extending up to the device enclosure, for example, all the way to the circuit board, on which an electrical connection can be made so that resilient member 396 serves also as the electrical contact to the electrode 398.

FIG. 10E illustrates different approaches to delivery of electrical stimulation. An electrical output requires at least two “poles” for delivery. A bipolar delivery occurs between two relatively closely spaced electrodes. For example, some of the designs shown above include first and second electrodes disposed, respectively, on the conchae caverna and conchae cymba; when electrical signal passes between two such electrodes, a bipolar output is generated. Line A, between electrodes X and Y in FIG. 10E can be understood as indicating a bipolar output. On the other hand, a monopolar delivery occurs between a first electrode positioned at a therapy site, and a remote electrode located away from the therapy target. Lines B (between electrode Y and a remote electrode, R) and C (between electrode X and a remote electrode, R) indicate monopolar therapy combinations. Multiple return electrodes can be used, for example to influence voltage fields and spread, focus or steer the outputs, such as shown in FIG. 10F where electrode X issue a therapy output with two return electrodes. The return electrode in FIGS. 10E and 1OF may be positioned at any desired distance, as indicated with the use of the broken line gaps. Each of these different types of therapy may be used in various examples. Some examples will omit the remote electrode, R, such as the designs shown above in FIGS. 2A-2E, 3, 4A-4B. FIG. 5, for example, shows a remote electrode 238, which may be a separately placed and/or positioned electrode, if desired, placed, for example, on the torso of the patient, on a limb, or elsewhere on the patient's head or neck, as desired. In one example, the therapy target is the auricular branch of the vagus nerve, and the remote electrode 238 may be placed on the loop 236, so that it contacts the back side of the ear and/or the side of the patient's skull near the ear. Monopolar therapy can be used instead of or in addition to bipolar therapy.

Other therapy modalities may not require paired neural stimulation elements. For example, as indicated in FIG. 10G, some stimulation elements, such as S1, can generate output stimulation that travels in a range of directions. Vibration/acoustic stimulation, as well as magnetic stimulation, may travel in this way, such that S1 may be understood as an electro-mechanical, or electro-magnetic transducer. Thermal therapy can be generated as well, and so S1 may instead be a thermal element such as a resistor that converts electrical current to heat. Some optical outputs provide collimated light outputs, such as light emitting diodes and/or vertical cavity surface emitting lasers, as illustrated at S2. A dispersing lens may be included as shown for S3, to provide a spread the output light energy; alternatively, a less directional light source can be used, as desired.

FIG. 11 is a block diagram for illustrative circuitry. The illustrative circuitry may be described as operational circuitry for the device, and would be contained in the housing as shown in any of the preceding examples. The device includes a controller 400. The controller 400 may take many forms, including, for example, a microcontroller or microprocessor, coupled to a memory 402 storing readable instructions for performing methods as described herein, as well as providing configuration of the controller for the various examples that follow. The controller 400 may include one more application-specific integrated circuits (ASIC) to provide additional or specialized functionality, such as, without limitation, a signal processing ASIC that can filter received signals from one or more sensors using digital filtering techniques. Logic circuitry, state machines, and discrete or integrated circuit components may be included as well. A controller 400 may take the form of a state machine, if desired. The skilled person will recognize many different hardware implementations are available for a controller. Likewise, the memory 402 can take any suitable form, including Flash memory, combinations of multiple memory types, etc.

The operational circuitry also includes a power supply block 404, coupled to a battery 406. The power supply block may include voltage step-up or step-down circuitry, or may include appropriate regulators, converters and the like, as well as smoothing circuitry as needed/desired to obtain power from a battery 406 and provide power at specified voltage/current for use in the controller 400 as well as the output circuitry shown at 410. One, two, three, four or more battery cells may form a battery 406; commercial off-the shelf button-type batteries may be used, or specialized versions may be developed and used. For example, three or four lithium-chemistry button batteries may provide 9 or 12 volts of power supply, allowing maximum currents in the device to stay relatively small (reducing heat), while generating sufficient headroom to provide desired current or voltage levels for therapy. Batteries may be replaceable, if desired. Rechargeable batteries could be used, whether removeable and rechargeable or by providing a recharging circuit as indicated at 408, in the device, where power can be transferred to a recharging circuit by use of an electrical port on the device, or by wireless transmission (inductive, RF, ultrasonic, etc.) to a transducer on or inside the device. An example may use an inductive loop coupled to a rectification circuit that in turn delivers current/power to the battery 406 for recharging, for example. A recharging case or cord, for example, can be used to enable recharging of the device or devices (for example, if provided in pairs, rather than as stimulation for a single ear). Some examples may include electrical contacts on the device for recharging in a recharging case/housing, if desired.

The power supply 404 may further include a dedicated voltage converter to provide, for example, a source for a current controlled output circuitry. In an example, an inductive or capacitive step-up circuit is used to store a 60-volt amplitude on one or more capacitors to provide headroom for a current controller output circuit using, for example, one or more current mirrors to control the output current. Suitable amplifier-based circuits may be used, instead, or any other desired circuit can be used. While inductive step-up circuitry can be used, capacitive converter designs may provide better MRI-compatibility and tend to be smaller and introduce less weight.

The output circuitry 410 may include a set of switches, such as an H-Bridge circuitry design, configured to provide alternating signal outputs. Square wave outputs may be used, and may be current controlled or voltage controlled, as desired. Non-square waves can be used as well, such as exponentially decaying, sinusoidal (in which case a resonant circuit can be included), triangle, ramped, etc. The output circuitry may be coupled to electrodes 430 for use in delivering electrical stimulation. Alternatively or in addition, one or more transducers 432 for use in issuing optical, mechanical, or magnetic stimulation outputs can be coupled to the output circuitry 410. The power supply 404 is configured to provide voltage step-up (such as a voltage multiplier using inducive or capacitive elements), allowing the output circuitry to shape and control the power signals issued to the electrodes 430 or transducer 432. The transducer 432 may also receive control signals from the controller 400 to manage, for example, output frequency of the transducer, depending on design.

A set of monitoring circuitry 412 can be included to monitor the operations of the output circuitry. For example, sample/hold circuits can be used to determine the voltage while issuing current controlled outputs, allowing the impedance that output signals encounter to be tracked. If a voltage-controlled output is used, current monitors can be used to determine impedance as well. If an exponentially decaying output is provided, the slope of voltage on a capacitor used to output the signal may be determined by monitoring voltage change over time, to give a measure of impedance as well. High impedance, over a predetermined threshold for example (which may vary with device placement, and/or inclusion or exclusion of hydrogel or other tissue interface enhancers) may be used to determine appropriate device placement and/or tissue contact. Low impedance, on the other hand, can indicate shorting between the output electrodes that could present a burning hazard to the patient, depending on other system controls.

The monitoring circuitry 412 can include, for example, a temperature sensor (such as a thermistor, resistance temperature detectors, thermocouples, and/or integrated circuit sensors) to monitor temperature at the tissue interface. If sensed temperature is below a low temperature threshold (for example, 25 C), the monitoring circuitry 412 may stop therapy due to sensed poor tissue contact, and if the sensed temperature is above another threshold (for example, 40 C), the monitoring circuitry 412 may stop therapy (or reduce therapy amplitude/intensity or change other therapy parameters) due to potential burn hazard due to, for example, device error, malpositioning, or amplitude settings that are too high. Other sensors may be included to monitor therapy output parameters, patient response, or patient characteristics to ensure therapy efficacy and/or prevent patient harm.

The monitoring circuitry 412 may also monitor battery status, including, for example, a current sensor or coulomb counter if desired to track actual battery use, or a voltage sensor to determine open, lightly loaded, or loaded output voltage of the battery 406 or individual cells therein. Battery usage may instead be tracked, for purpose of determine battery end of life/status, by the controller 400 using timers and therapy counting, as desired. Monitoring circuitry 412 may observe characteristics of the output circuitry to, for example, ensure that the power supply 404 is providing sufficient voltage step-up to allow therapy to be delivered as desired.

The memory 402 may store controller-readable instructions for operating the device in any suitable form, and can also store operating data, including time spent in pause, on/off or other operational data. Operational data may include temperature or impedance data, if desired, or any other sensed parameters or signal.

The controller is also coupled to what may be termed input-output devices, including any buttons 420 on the device, and/or the lights or speakers 422 described above. A screen or touchscreen may be used instead or as well as those items shown. Some systems may optionally include an RF circuit block 424, including, for example and without limitation, Bluetooth, WiFi, and/or any wireless communications circuitry (antenna, driver, crystal/resonator, etc.) for performing wireless communication with a separate device. For example, a smartphone operating an application may communicate via Bluetooth with the device to control any characteristic of therapy (duration, on/off, repetition rate, amplitude, pulse width, type, etc.) and/or to obtain therapy data (impedance, usage, etc.).

General purpose devices may communicate with the therapy system if desired, using for example an application operating on a smartphone, tablet, or computer. A “programmer” maybe used instead of a general-purpose device, where a programmer is a dedicated device configured for use with the stimulation system. In some examples, to simplify use in an emergency context or even in the ICU, a dongle may be provided that carries programming circuitry (communications and stored software instructions) for communicating with an programming a therapy device, wherein one end of the dongle can be plugged into, for example, a universal serial bus port (or any other port) on a tablet, smartphone or computer; once plugged, the software on the dongle can launch an application on the device for programming the therapy system, so that the physician/nurse or other medical user does not need to download any software to control the system.

Communication may be used to modify therapy settings, upload new software to the device, and/or to download therapy or other usage data. Device status, such as battery capacity, may be communicated. Communication may also be used to turn the device on or off, if desired, rather than relying on a button or other actuatable component on the device and/or device housing.

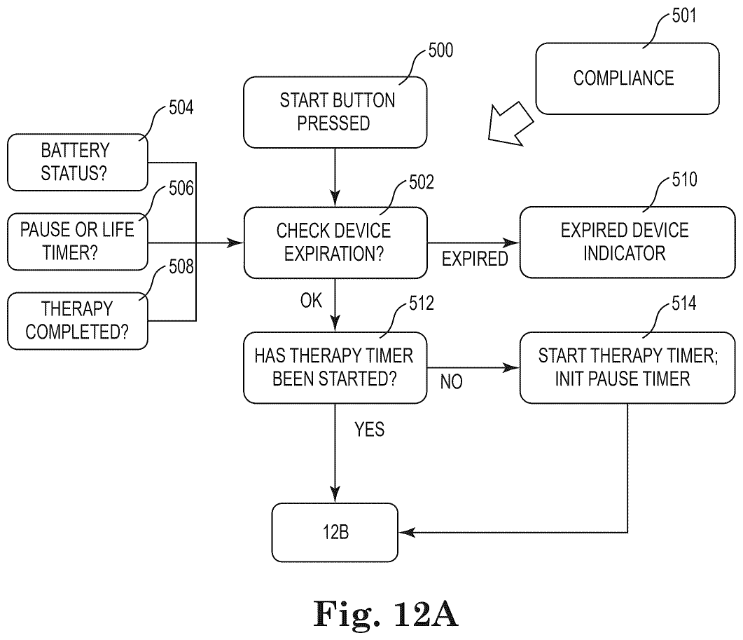

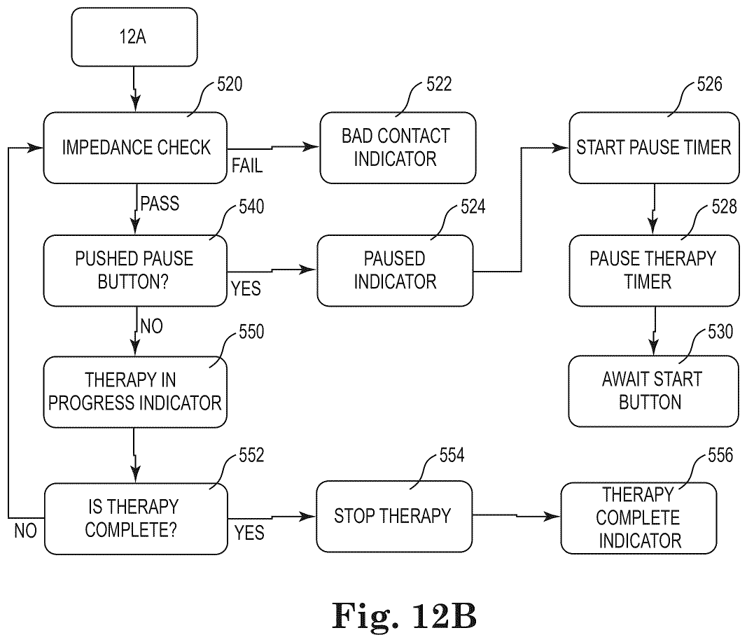

FIGS. 12A-12B show a block process flow diagram of an illustrative method. A relatively comprehensive method is shown; some other or alternative examples may omit one or more blocks/steps, or may replace the illustrative steps shown with other steps. At block 500, a user or a person helping the user (a nurse, physician, caretaker, etc.) presses the start button on the device. The device controller then uses stored instructions to execute a method as shown and described herein. First, the device controller checks device expiration, as indicated at 502. This may include determining battery status at 504, such as by comparing a measured battery voltage (open circuit or lightly loaded in some examples) to a battery voltage threshold. If the battery voltage is below the threshold, this indicates a low battery condition, and the controller will then activate an expired device indicated at block 510. If the expired device indicator is activated at 510, the method stops.

Checking device expiration at 502 also includes, in some examples, observing the status of a pause or life timer. A pause timer may indicate an amount of time since a therapy was initiated. A life timer may indicate an amount of time since the device was activated. One, the other, or both timers can be used in any given implementation. If either timer has exceeded a predetermined threshold, the controller determines the device has expired, and the controller will then activate an expired device indicated at block 510. Once the expired device indicator is activated at 510, the method stops.

Checking device expiration at 502 also includes, in some examples, determining whether a therapy regimen has been completed, as indicated at 508. Once the predetermined therapy regimen for the device has been completed, the controller determines the device is expired, and the controller will then activate an expired device indicated at block 510. Once the expired device indicator is activated at 510, the method stops. For example, a therapy regimen can be predetermined to provide a 20 minute duration therapy, once daily, for up to two weeks. For purposes of block 502, if more than two weeks, plus one to seven days to allow for therapy pauses, has occurred since device activation, this may be treated as exceeding the life timer, or if all the therapy steps of the regimen have been performed, this may be treated as completing the therapy, or if a pause of more than a set duration (72 hours, for example) has occurred, this can be treated as exceeding the pause timer. In some examples, a dual pause timer threshold is present. For example, if a first preset period (one to four hours, for example) is exceeded since a therapy pause, a therapy dose (for example a 20 minute session may be the therapy “dose”), the therapy dose can be re-started; if a second preset period (48 to 96 hours, for example) is exceeded, this can be treated as if the therapy regimen has been interrupted or stopped, and the device is treated by the controller as expired.

If the method passes block 502 without device expiration, the controller determines whether the therapy timer has been started at block 512. If the therapy timer has not been started, the method goes to block 514 and starts the therapy timer. At block 514, the therapy pause timer may be initialized or reset, to ensure that it starts from zero if the therapy timer is also starting from zero. The method then proceeds to FIG. 12B.

In FIG. 12B, an impedance check is performed at 520. The impedance check may be performed by outputting a therapeutic or non-therapeutic pulse/waveform to obtain an impedance measurement and check the contact between the electrodes and the patient's skin. For example, a non-therapeutic pulse can have a shorter pulse width, or a shorter amplitude, than a therapeutic pulse width so that the impedance can be checked without outputting a relatively higher energy therapy pulse. As noted, a therapy pulse can be used instead. The impedance, once obtained, is compared to at least a high impedance threshold, which, if exceeded, indicates poor or no contact with the patient tissue. Impedance may be compared to a low impedance threshold as well, if desired, to ensure there is no shorting of the output electrodes, in which case therapy may be ineffective if it does not reach the target tissue. If the impedance is out of range (too high, or too low, for example) the controller activates the bad contact indicator as indicated at 522. Therapy is paused as indicated at 524, and the pause timer starts at 526. The therapy timer is paused at 528, and the system awaits actuation of the start button. Here, the end state is that therapy is paused with the bad contact indicator active, until the user adjusts device position and depresses the start button, which will return the method to block 500.

While impedance can be used for systems having electrodes for issuing electrical therapy, other system placement checks can be performed. In an example, a temperature sensor is checked to confirm an appropriate sensed temperature range (for example, 25 C to 40 C). For example, a temperature sensor can be positioned on the anchor arm, or adjacent to a stimulation element, such as a transducer. When the device is positioned against patient tissue, the sensed temperature should be near the temperature of tissue, typically in the range of 36 C to 37 C; the broader range of 25 C to 40 C may be used to allow for the device to activate promptly after positioning on tissue, in which case the temperature sensor may lag for seconds or even minutes as the surfaces and materials of the device warm up to body temperature in response to body contact. Contact or positioning may be determined using other approaches, such as having a pair of non-therapy, sensing-only electrodes positioned to sense tissue contact. If an optical output is used, an optical sensor may be positioned to observe reflected light from patient tissue, thereby confirming contact if the reflected light matches an expected bandwidth and/or intensity, for example. These examples are intended to be illustrative and not limiting.

If the impedance check is passed at 520, the method determines whether the pause button has been depressed at block 540. If so, the method includes activating the paused indicator at 524, and then follows the rest of the steps at 526, 528 and 530. Here, however, the end state is that the therapy is paused with only the paused indicator active, until the user depresses the start button, which will return the method to block 500.

At block 550, having passed the checks at 520 and 540, therapy is activated if not already active, and the device issues at least one output pulse. As this occurs, the therapy progress indicator is activated. After at least one output pulse is generated, the method proceeds to block 552, to determine whether therapy is completed. Therapy complete may be determined using a time since therapy started, a total time of therapy active, or a quantity of pulses delivered, or some other measure as desired. If therapy is not completed at block 552, the method reverts to block 520 and re-runs the impedance check; on such a reversion, in some examples, impedance determined during therapy pulse outputs is used, rather than any test pulses as described previously, so that therapy does not have to be interrupted. Alternatively, the method may return to block 520 at periodic intervals (every 1 to 15 seconds, for example) when therapy can be interrupted for a test pulse to determine impedance. As noted, non-impedance-based therapy monitoring or tissue contact monitoring can be used instead.

If therapy is complete at block 522, the method stops the therapy outputs and ceases all clock/timer activity at 554, and sets a therapy complete indicator at 556. A stored bit may be set as well to indicate therapy complete, so that actuation of the start button at any future time will result in a device expiration in the step at 502 in FIG. 12A.

For a recurring therapy method, there may be a shell outside of that shown in which a regimen of therapy is provided by executing a method as shown in FIGS. 12A-12B daily. The checks performed at block 502 may include a regimen complete check, as desired.

Returning to FIG. 12A, in some examples, a replaceable battery or rechargeable battery is used; if so, the battery status 504, if failed, may yield a battery alert indicator being activated, rather than expired device. Additional monitors and checks may be included. A watchdog timer, as well as other failure monitors, can be used to set a failure or device expired bit in memory, causing the device to be marked as expired.

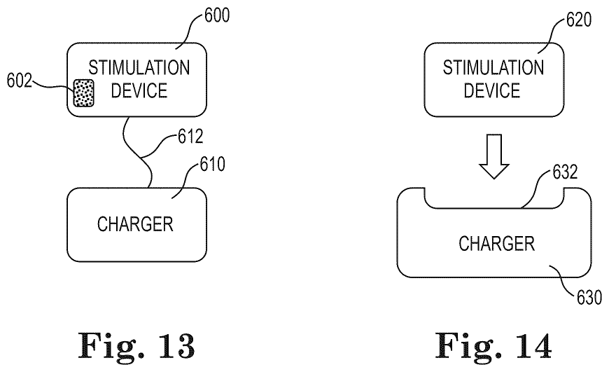

Throughout both FIGS. 12A and 12B, compliance 501 can be monitored in several ways. The device itself may carry, for example and without limitation, a barcode or quick response (QR) code readable or scannable by a mobile device, such as a smartphone, allowing device registration. Device registration can be used to activate the device remotely, to provide a code allowing device activation to a mobile device, and/or to track device activation and/or usage by the manufacturer or a health care provider. For example, in the intensive care unit (ICU) or other medical care context, a nurse, technician, physician or other person may scan a QR code when a device is applied to a patient. A device readable item (QR code for example) is illustrated on a stimulation device in FIG. 13, at 602, for example. Any suitable positioning, including on external packaging of the stimulation device or on the device itself, can be used as desired. The QR code may be positioned on a removable tab 318 as shown in FIG. 7 so that code scanning only takes place after the device has been activated, preventing erroneous or early activation.

Some examples may make use of a device logging function internal to the device electronics. For example, a log of usage and/or measured data may indicate (including with time stamps) when and how the device has been used by recording voltages and/or current delivered. Such a log may be stored in suitable manner in device memory. Any measurements the device takes including, for example, measured temperature, impedance and/or other data may be stored in a log file as well. Log files may be accessed and/or retrieved from a device by the use of wired transfer (such as via a plug-in device to a charger as in FIG. 13, to a smartphone or computer, for example) and/or wireless transfer such as by cellular, Bluetooth, WiFi, optical or other communications modality. All or selected portions of logged data may be used to track compliance and/or to observe device operations for device quality or any other desired purposes.

The process flow in FIG. 12B may be adjusted to use an interrupt, rather than a recurring check, on the user depressing or actuating the pause/start button. Thus block 540 may be used to interrupt any of the other process steps in response to user action, as desired. For example, block 550 may be a therapy on block, operable for a period of one to thirty seconds, during which actuation of the pause button would work as an interrupt. Periodically, then, the system would perform the impedance check. In other examples, the impedance check is performed after each delivered therapy pulse, if desired.

The device and system may be configured for a variety of use cases. For example, the wearable vagus nerve stimulation can be used in conjunction with pharmacological interventions to treat sepsis in ICU patients. By targeting inflammation including use of vagus nerve stimulation, the system it can help modulate the immune response and potentially improve outcomes in patients with severe sepsis.

In another example, in patients with acute respiratory distress syndrome (ARDS) in the hospital setting, wearable vagus nerve stimulation can be utilized alongside mechanical ventilation and anti-inflammatory medications to reduce lung inflammation and improve oxygenation. This combined approach may enhance the overall management of ARDS and potentially speed up the recovery process.

In another example, for patients with severe pneumonia requiring intensive care, wearable vagus nerve stimulation can complement antibiotic therapy and respiratory support by targeting systemic inflammation. By regulating the inflammatory response, this adjunctive therapy may help in reducing the severity of pneumonia and preventing complications in critically ill patients.

In another example, in the management of inflammatory bowel disease (IBD) exacerbations in hospitalized patients, wearable vagus nerve stimulation can be used along with corticosteroids and immunosuppressants to control intestinal inflammation. This combined treatment approach may offer a novel strategy to alleviate symptoms and promote mucosal healing in patients with severe IBD flares.

In another example, wearable vagus nerve stimulation can be combined with pain management techniques in post-operative ICU patients to mitigate surgical inflammation and improve recovery outcomes. By targeting the inflammatory cascade, this adjunct therapy may aid in reducing post-operative complications and enhancing the overall healing process in critically ill surgical patients. In addition, again for the post-surgery context, wearable vagus nerve stimulation can be utilized post-operatively to enhance bowel motility by delivering targeted electrical impulses to the vagus nerve, promoting gastrointestinal motility and reducing the risk of post-operative ileus.

In another example, a wearable vagus nerve stimulation device can be used in conjunction with remote monitoring systems to continuously track the patient's heart rate, blood pressure, and other vital signs. By integrating real-time data from the device with the digital monitoring platform, healthcare providers can quickly identify any signs of worsening heart failure and intervene promptly to prevent readmission.

In an example, wearable vagus nerve stimulation can be used in combination with traditional pharmacological treatments for Congestive Heart Failure (CHF) to reduce readmission rates. By incorporating vagus nerve stimulation into the patient's treatment plan, the device can potentially improve heart function, reduce inflammation, and enhance autonomic balance, leading to better overall outcomes and decreased risk of hospital readmission.

FIGS. 13-14 show stimulation devices with chargers. For a patient to use a device in a chronic sense, the power supply must be either replaceable (such as with replaceable batteries) or replenishable. A rechargeable stimulation device may be useful in any context. In FIG. 13, a stimulation device 600, carrying a QR code marker 602, is shown connected to a charger 610 using a wire 612. The connection may use standard connectors, such as uniform serial bus (USB) connectors, micro-USB, etc., or may be a special purpose connector 612 to prevent unauthorized use or modification of the stimulation device 600, if desired. The charger 610 may be battery powered or may use wall power, as desired. The stimulation device 600 may remain positioned in the ear of a patient during charging, or may be removed. Another example is shown at FIG. 14. Here the stimulation device 620 is received in a charger 630, having a depression or cradle 632 for receiving the stimulation device 620. Electrical connectors can be provided in the cradle 632, positioned to align with electrical connections on the outside of the stimulation device 620 (such as any of the electrodes shown above, or using connectors adapted specifically for charging). Other modes of power transmission can be used, including inductive, RF, optical, etc., as desired.

In each of FIGS. 13-14, data transmission can be performed while charging takes place. For example, logs of therapy utilization and/or device status may be transferred to the charger 610, 630, which may in turn transmit any received data to a central database by any suitable communications mode, such as over the internet, cellular, etc. Likewise, therapy firmware in the stimulation devices 600, 620 may be updated, or settings modified, as desired. The chargers 610, 630 may be connectable to additional devices, such as a smartphone operating a dedicated application for the purposes of software updating, logfile reading/review, and/or parameter modification, if desired.

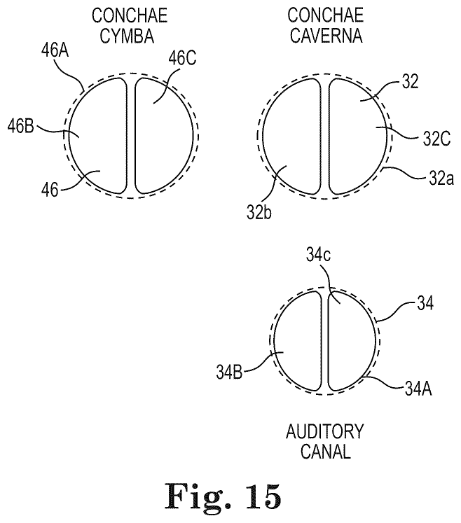

FIG. 15 illustrates various electrode configurations that can be used, as desired. Electrode structures are shown at 32, 34, and 46, corresponding to the electrodes that can be used in systems as shown above in FIGS. 2A-2E. Various examples of system electrode configurations may be achieved. Electrode structure 34 would be positioned on the anchor arm, and is omitted in some examples. The electrode structure 32 may be a single electrode 32a, or may include more than one electrode, such as electrodes 32b, 32c (more than two may be used, if desired). Likewise, the electrode structure 46 may be a single electrode 46a, or may include more than one electrode, such as electrodes 46b, 46c (more than two may be used, if desired). When present, the electrode structure 34 may be a single electrode 34a, or may include more than one electrode, such as electrodes 34b, 34c (more than two may be used, if desired). Various combinations are contemplated:

Electrode structure 32 may include two electrodes 32b, 32c, with therapy delivered between those two electrodes 32b, 32c only, directing therapy to the conchae caverna. Other electrodes may be omitted, or may be present but inactive, or may delivered a separate waveform. Electrode structure 46 may include two electrodes 46b, 46c, with therapy delivered between those two electrodes 46b, 46c only, directing therapy to the conchae cymba. Other electrodes may be omitted, or may be present but inactive, or may delivered a separate waveform.

Each of electrodes 32b, 32c, 46b, 46c may be included in some examples, and therapy may be delivered in sequential anode/cathode pairs, for example as shown here:

| Anode | Cathode |
|---|---|
| 32b | 46b |
| 32c | 46b |
| 32b | 46c |
| 32c | 46c |

Therapy may start at the top row and proceed to each successive row. After one round of such therapy, the sequence may be repeated with opposite polarity, for example. Other combinations and/or sequences can be used. As the skilled person will understand, this approach may require multiple sources in the electronics of the device, providing multiple, independent signals to control individual electrodes. Some other examples may have one electrical source and a plurality of switches to direct therapy signals as desired. Some examples may have multiple electrical signal sources and a set of switches arranged to multi-plex the output signals as desired.

With a larger number of electrodes, additional flexibility is enabled allowing the electrical field applied to the underlying tissue to be shaped or tailored as desired. Groupings of electrodes may be electrically connected to form larger or smaller effective stimulation areas. Individual or grouped electrodes may be independently controlled to provide varying levels of stimulation so as to shape activation fields to location or depth to preferentially activate underlying tissue, or to avoid or suppress activation of underlying tissue. In some examples, stimulation intensity can be adjusted to account for electrode position/proximity and/or side. For example, larger currents can be delivered with ganged-together electrodes with less concern regarding patient comfort. Also, varying frequencies of stimulation between electrodes may be used to activate, inhibit, or avoid stimulation of underlying tissue by creating interacting activation fields, like beat frequencies, or inferential therapy.

The circuitry in the stimulation device may include multiple outputs that allow for independent control over each electrode and/or plural electrode pairs, if desired, to allow multiple waveforms to be delivered at the same time. For example, a sinusoidal first stimulation signal issued between electrode 32b and electrode 46b at 40 Hz could be output at the same time as a second stimulation signal generated at 30 Hz using electrode 32c and electrode 46c, resulting in a 10 Hz beat frequency arising within the patient tissue. Other “beat” related approaches or interferential signals may be used instead or in addition to these examples.

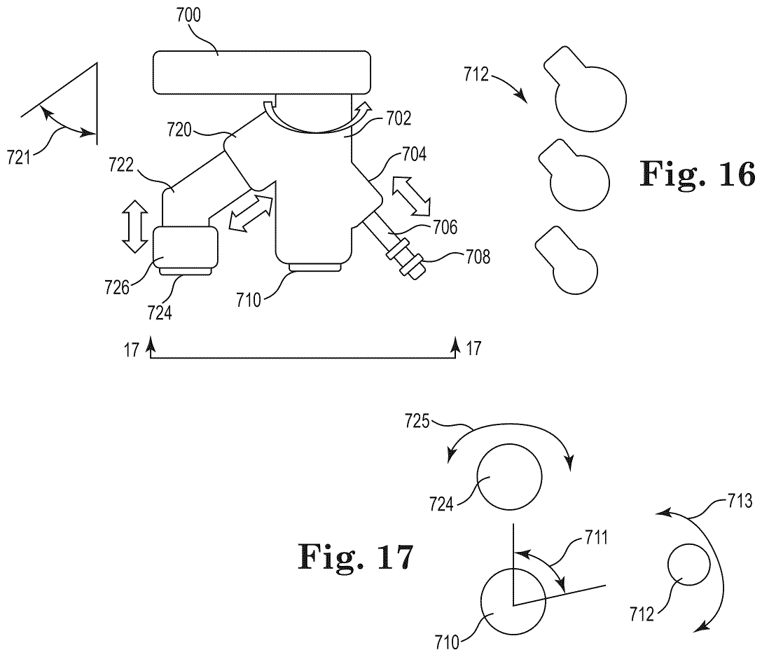

FIGS. 16-18 illustrate further structures. In FIG. 16, a wearable vagus nerve modulation device 700 includes an extending structure 702 which carries an anchor arm 704, having an extending anchor arm 706 moveably mounted therein as indicated by the arrow. Ridges are provided as shown at 708 for holding a removable tip thereon, to anchor in the auditory canal of the user's ear. An electrode or transducer (optical, sonic, magnetic, thermal, etc.) is provided as stimulating element 710. When placed, the stimulating element 710 may be placed against the user's skin in the conchae caverna. A plurality of tips 712, in a range of sizes if desired, are provided with the device. Tips 712 may come in various shapes or sizes to allow different users to select a best fit. Tips 712 may be replaceable, as the position in the auditory canal may lead to wax build up, for example, making occasional or periodic replacement useful.

The extending structure 702 also carries a stimulator extension 722 which can be extended or retracted relative to a receiver 720, such as by including a spring-loaded structure, as indicated by the arrow. The stimulator extension 722 forms an angle 721 relative to the axis of the extending structure 702, the angle being, illustratively, in the range of about 30 to about 60 degrees; in an example, the angle 721 is about 45 degrees. A stimulating element 724 is positioned on a carrier 726, which may be a generally hollow piece that can slide over the stimulator extension 722, as indicated by the arrow. Positioning may again be spring loaded, if desired. This design has a single extending structure 702 relative to the main body of the device 700.

The extending structure 702 may be rotatable (at least partly) if desired, allowing the main body to directed, vertically, horizonal, or at an angle therebetween when placed on the patient. For example, if a patient is in a recumbent position in the ICU, as opposed to being ambulatory outside of the ICU, different positioning may be desired, so the rotation of the extending structure may be used to adjust for comfort and secure positioning. In some examples, the receiver 720 is rotatable relative to the anchor arm 704, for example, allowing different angles to be defined therebetween, if desired.

FIG. 17 shows the orientation of actuators or electrodes of FIG. 16. It may be noted that FIG. 16 illustrates the location of the anchor arm 706 relative to the stimulator extension 722 at an angle so that the stimulator extension 722 can be observed in one drawing. FIG. 17 illustrates these angles with a bit more clarity. The stimulating element 710, along with the stimulating element 724 and the anchor arm at tip 712 form an angle as shown at 711. The positions of stimulating element 724 and tip 712 are adjustable as illustrated with arcs 713 and 725, so that the angle 711 can be varied in the range of about 60 degrees to about 135 degrees, or more or less. The angle 711 may be, for example, about 90 degrees, if desired. The angle 711 can be adjustable if desired. In some examples, angle 711 is instead a fixed angle.

In some examples, textured, ridged, disk, or bulbous shapes (or combinations thereof) may be used to aid in securing the device in place by including such shapes on the anchor and/or an extending structure. For example, the three elements 710, 712, and 724 shown in FIG. 17 each represent touch points to tissue of a patient. Any one, two or all three of these touch points can include a shape (such as bulbous or disk-shape), ridges, texture or roughening that discourages or prevents passage along or past tissue or a tissue ridge or layer, such as the helix, antihelix, helical crus, intertragal notch, tragus, and/or anti-tragus. Such shape, ridges, texture or roughening may be applied on all sides of any of the three touch points, or only along an outer edge or tissue-contacting side thereof, as desired. By outer edge, the intent is to indicate the portion of any anchor or extending structure that would press against tissue to hold the device in a desired position.

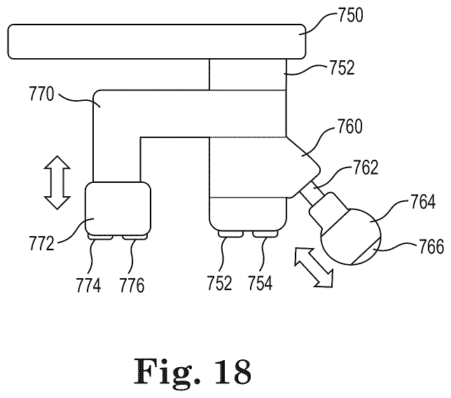

FIG. 18 shows another example. Here, the wearable vagus nerve modulation device has a main body 750 and an extending structure 752. The extending structure 750 terminates with electrodes or transducers (optical, sonic, magnetic, thermal, etc.) provided as stimulating elements 752, 754, which may be positioned over the conchae caverna, as desired. An anchor extension 760 extends off at an angle from the extending structure 750, and carries an anchor arm 762 adapted to receive an anchor 764 for placement in the auditory canal. The anchor 764 can be replaceable and/or may come in various sizes and shapes, and further may include one or more electrodes or transducers (optical, sonic, magnetic, thermal, etc.) thereon, shown as stimulating element 766.

A stimulator extension 770 is also provided from the extending structure 752, extending laterally therefrom and having a carrier 772 for one or more stimulating elements 774, 776, which can be electrodes or transducers (optical, sonic, magnetic, thermal, etc.) as desired. The stimulator extension 770 is at more or less a right angle relative to the extending structure 752 in the illustrative example, as contrasted with the use of an angle in the range of about 30 to about 60 degrees for the stimulator extension 722 of FIG. 16. The use of a sharper angle may make positioning easier relative to anatomical structures of the ear, such as avoiding interaction with the crus of helix.

The anchor arm 762 may be extendable/retractable, as indicated by the arrow. Likewise, the carrier 772 may extend or retract over the stimulator extension 770, as indicated by the arrow. Optionally, in some examples, the stimulator extension 770 and/or the anchor extension 760 may be rotatable relative to the extending structure 752, allowing repositioning and adjustment if desired. Still further, the extending structure 752 may itself be rotatable, in some examples, relative to the main body 750. Rotation of the extensions and/or structures relative to one another can be omitted in other examples.

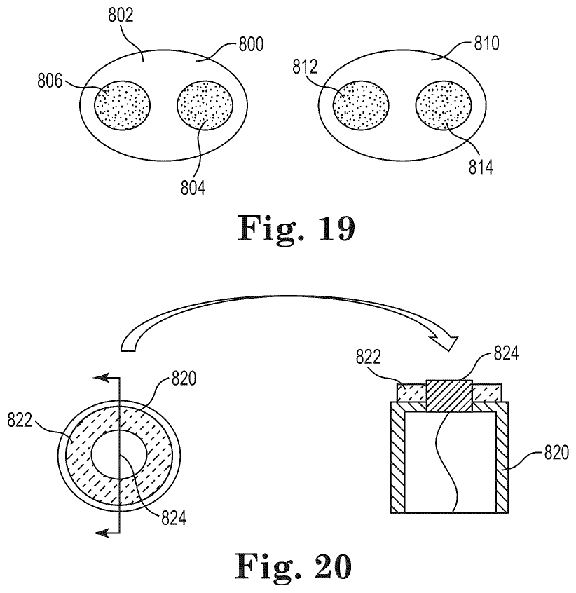

FIGS. 19-20 show illustrative gel pad designs. In FIG. 19, the gel pads are provided on the device for conducting electric (or other) signals to the skin. For example, a metal electrode placed on dry skin can encounter a larger impedance, thus attenuating therapy signals, than an electrode placed over a conductive liquid or gel on the skin. In the example of FIG. 19, a first extending structure 800 may include two electrodes, each of which may be recessed relative to a surface 802. Gel pads 804, 806 reside on the electrodes in each associated recess and include or are made of a conductive and biocompatible material such as, but not limited to, a hydrogel. The recess helps to hold the gel pads in place, and prevents, for example, the gel pads 804, 806 migrating off of the electrodes and/or coming into contact with one another, either of which could reduce therapy efficacy by increasing tissue interface losses with added impedance (in the case of gel pad migration), or by shunting current (in the case the gel pads come into contact or close proximity with one another). A second extending structure 810 uses a similar recessed electrode structure and gel pads 812, 814. The gel pads 804, 806, 812, 814 may be a custom consumable for the system, and may be replaced with each use (in the event of home use) or may be left in place for a single-use device over the course of a therapy session or regimen lasting hours to weeks. Use of the recessed electrode is optional.

FIG. 20 shows an alternative example. Here, the extending structure 820 carries a pad 822 that surrounds the stimulation element 824. In this case the pad 822 is used to help stick to the skin of the patient, and may have an adhesive backing for placement against the extending structure 820, surrounding the electrode 824 as shown in the inset side-section view. The pad 822 may be a custom consumable for the system, and may be replaced with each use (in the event of home use) or may be left in place for a single-use device over the course of a therapy session or regimen lasting hours to weeks. If the device uses an electrode as the stimulation element 824, the pad 822 may be a non-conductive substance, if desired, so that the electrical therapy output says focused at the location of the stimulation element 824. The pad may, for example, be a substrate of any suitable material, including natural materials (wool, cotton, silk, etc.) having an adhesive on each side, or a synthetic material, such as but not limited to polymers, again with an adhesive on each side thereof. Pad 822 may comprise a gel or other semi-liquid material.

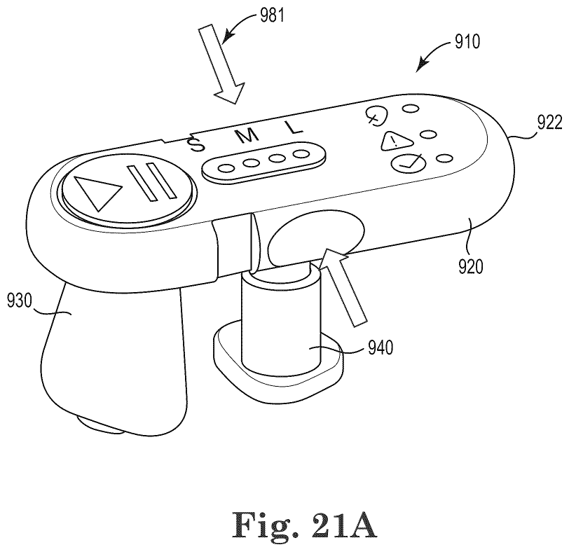

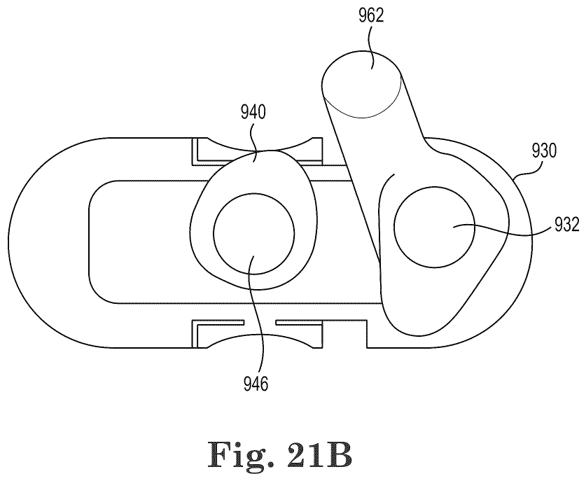

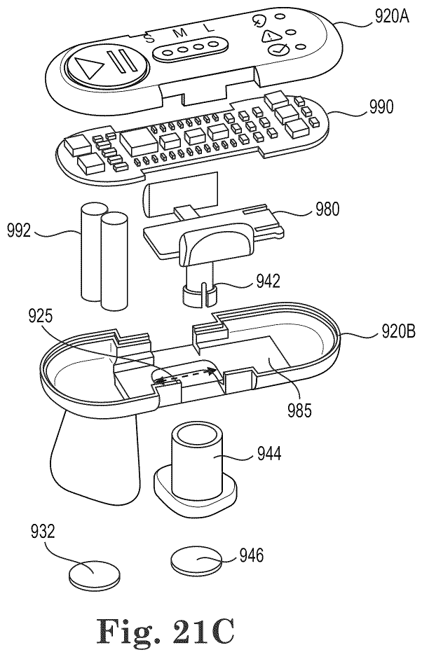

FIGS. 21A-21C are views of a fifth wearable vagus nerve modulation device 910. FIG. 21C shows an exploded view of device 910. As an example, housing 920 may comprise molded pieces 920A, 920B assembled together. In this example, the first molded piece 920A may be an upper lid and a second molded piece 920B may have a lower container to which the upper lid attaches, collectively forming housing 920. In this example, the first extending structure 930 is shown to be integrally molded as part of the lower container 920B. Molded into the lower container 920B is an internal channel 985 configured to receive carriage 980. In some examples, the carriage 980 and the upper portion 942 of the second extending structure 940 may be molded together as a singular piece for positioning into the internal channel 985.

The lower container 920B also includes an elongated aperture 925, situated between the first extending structure 930 and the first end of the housing 922. The upper portion 942 of second extending structure 940 protrudes through the aperture 925 away from the housing 920. Once the upper portion 942 is in place, the lower portion 944 of the second extending structure is secured over the upper portion 942. The position of the carriage 980 in the internal channel 985 is adjustable, and by virtue of the upper portion 942 of the second extending structure being carried on the carriage 980, the position of the second vagus nerve stimulating element 946 at the end of the second extending structure is also adjustable. That is, the space between the first vagus nerve stimulating element 932 and the second vagus nerve stimulating element 946 can be adjusted. During placement of the device in the ear of the patient, the carriage position can be adjusted such that the second vagus nerve stimulating element 946 is positioned on a conchae cymba while the first vagus nerve stimulating element 932 is positioned desirably at the conchae caverna.

In some examples, the second vagus nerve stimulating element 946 may be positioned at the conchae caverna. In this configuration, the second vagus nerve stimulating element 946 is not part of the second extending structure 940, but rather integrated into housing 920 or the first extending structure 930, enabling the second vagus nerve stimulating element 946 to be in contact with different areas of the conchae once device 910 is in position. In this example, the second extending structure 940 remains useful for stabilizing and securely attaching the vagus nerve modulation device 910 to the ear, regardless whether an electrode is carried thereon.

In some examples, the user may adjust the positioning of the second vagus nerve stimulating element 946 by manipulating carriage 980 from the outer face of device 910. By enabling adjustment from the outer face of the device 910, device 910 does not have to be removed from its previously secured. This form of manipulation also enables precise adaptation to individual ear anatomies, without compromising the stability of the device 910 placement as previously positioned. Adjustment of the carriage 980 in the internal channel 985 may be done manually or via a spring-loaded mechanism. In the manual configuration, users may adjust the positioning as needed including, but not limited to, using their fingers, tabs, hooks, loops, or pivoting levers. This adjustment can be done either before or after device 910 has been placed in the ear.

In some examples, the carriage 980 includes indentations or roughened surface to guide the user in adjusting the device and enhance grip. Including the indentations or roughened surfaces may be especially useful in the instance where the user that is manipulating the carriage 980 from the outer face of the device 910 after device 910 has been securely attached and the device 910 is no longer visible to the user. In the examples that use a spring-loaded mechanism, the mechanism can be compressed during device 910 placement in the ear and subsequently released to expand, effectively securing the device 910 in the ear. This expansion creates a counterforce against the conchae, so that the first extending structure 930 is in contact against tragus anti-tragus, ensuring a snug fit. In some examples, the spring-loaded mechanism incorporates a latch system, allowing the device 910 to be locked in various positions between fully compressed and fully extended states. This allows further customization the fit and positioning of device 910 in accordance to the user's ear anatomy and comfort preferences, while maintaining the device's stability and effectiveness.

In the illustrated example, the internal channel 985 overlaps with the aperture 925, to allow the second extending structure 940 to protrude through the lower container 920B when the carriage 980 is correctly positioned. The internal channel 985 exceeds the length of the aperture 925, providing a guided pathway for the carriage 980 to slide within the lower container 920B. As a consequence of such configuration, the range of the carriage 980 movement is limited to the extent that the second extending structure 940 can moveably slide within the bounds of the elongated aperture 925. This range of movement is illustrated by the arrow in FIG. 21C. By limiting such movement to the range allowed by the elongated aperture 925, manufacturing and assembly are made easier than if the housing had two components which slide together and apart, as controlling the maximum extent of movement is relatively simple. In an alternative example, rather than an aperture as shown, the overall housing may have first and second components that mate together in sliding fashion to allow the length of the housing itself to be varied.

This mechanism enables precise adjustment of the second extending structure's 940 position, ensuring optimal placement of the vagus nerve stimulating element on the conchae cymba without the need of the user to remove the device from the ear. The second extending structure may be of variable length, using, for example, a spring loaded or otherwise adjustable connection between the upper portion 942 and lower portion 944.

The circuit board shown at 990 has been specially shaped to accommodate the design, providing an elongated “bone” shape with first and second larger ends and a narrower middle portion, as can be seen. A more standard shape (simple rectangle, or rectangle with curved ends) may be used if desired. In some examples, the carriage 980 may engage with a toothed strip of material, so that a desired length can be selected and locked by, for example, compressing the upper portions of the carriage inward as shown at 981 in FIG. 21A.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “a” or “an” are used, as is common in patent documents, to include one or more than one, independent of any other instances or usages of “at least one” or “one or more.” Moreover, in the claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described above. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic or optical disks, magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72 (b), to allow the reader to quickly ascertain the nature of the technical disclosure. It is submitted with the understanding that it will not be used to interpret or limit the scope or meaning of the claims.

Also, in the above Detailed Description, various features may be grouped together to streamline the disclosure. This should not be interpreted as intending that an unclaimed disclosed feature is essential to any claim. Rather, innovative subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the protection should be determined with reference to the appended claims, along with the full scope of equivalents to which such claims are entitled.

What is claimed is:

1. A method of treating a patient comprising:
administering to the patient a medication;
observing an inflammatory response or inflammation-related side effect occurring after administering to the patient the medication; and
in response to the observing, delivering a vagus nerve therapy to the patient to alleviate the observed inflammatory response or inflammation-related side effect

2. The method of claim 1, wherein the vagus nerve therapy is delivered transcutaneously at a location on the patient to stimulate an auricular branch of the vagus nerve.

3. The method of claim 1, wherein the vagus nerve therapy is mechanical.

4. The method of claim 1, wherein the vagus nerve therapy is optical.

5. The method of claim 1, wherein the vagus nerve therapy is electrical.

6. The method of claim 1, wherein the medication is a chemotherapy agent.

7. The method of claim 1, wherein the medication is an immune checkpoint inhibitor.

8. The method of claim 1, wherein the medication is a hormonal treatment.

9. The method of claim 1, wherein the medication is an antipsychotic medication.

10. The method of claim 1, wherein the medication is a GLP-1 Receptor Agonist.

11. The method of claim 1, wherein the medication is a treatment for Alzheimer's disease targeting amyloid plaques.

12. The method of claim 1, wherein the medication is a Chimeric Antigen Receptor T-cell therapy.

13. The method of claim 1, wherein the medication is a diabetes drug.

14. The method of claim 13, wherein the medication is a thiazolidinedione drug.

15. The method of claim 13, wherein the medication is an SGLT2 Inhibitor.

16. The method of claim 13, wherein the medication is a DPP-4 Inhibitor.

17. The method of claim 13, wherein the medication is sulfonylurea.

18. The method of claim 13, wherein the medication is metformin.

19. A method of treating a patient comprising:
administering to the patient a medication, the medication having potential for inflammation related side effects; and
in response to the administering, initiating a regimen of vagus nerve therapy to the patient to prevent or alleviate the inflammation related side effect.

20. A method of treating a patient, comprising:
administering to the patient a medication, the medication having potential for inflammation related side effects;
after administering the medication at a first dosage, changing the dosage and administering the medication at a second dosage; and
in response to changing the dosage and administering at the second dosage, initiating a regimen of vagus nerve therapy to the patient to prevent or alleviate the inflammation related side effect.
